SEQUENCE LISTING (39 sequences)

SEQ ID NO: 1 - DNA, 79 bp, synthetic construct
  source 1..79: mol_type = other DNA
tagggaattcgtcgacggatcccgtggcgtctgcaacggaaaagaatttatcttgtcctgcaggtcgacgcatgcgccg

SEQ ID NO: 2 - DNA, 82 bp, synthetic construct
  source 1..82: mol_type = other DNA
ctgggaaaaccaccttcgtgatcatggtatctcccctgccaggtaagtataaaaaaaaaatatgacagaactagacactctt

SEQ ID NO: 3 - DNA, 82 bp, synthetic construct
  source 1..82: mol_type = other DNA
cgaacgcagtcccccactaccacaaattatgcagtcgagtttcccacattaaaaaaaaaatatgacagaactagacactctt

SEQ ID NO: 4 - DNA, 64 bp, synthetic construct
  source 1..64: mol_type = other DNA
tcttcctcgtggttttcggtgctctacacgttaaaaaaaaaatatgacagaactagacactctt

SEQ ID NO: 5 - DNA, 52 bp, synthetic construct
  source 1..52: mol_type = other DNA
ctccccaatacggagagaagaaaaaaaaaatatgacagaactagacactctt

SEQ ID NO: 6 - DNA, 79 bp, synthetic construct
  source 1..79: mol_type = other DNA
gttctaatctgccctccggaggaggaacaggtaaggattatcccaccaaaaaaaaaatatgacagaactagacactctt

SEQ ID NO: 7 - DNA, 50 bp, synthetic construct
  source 1..50: mol_type = other DNA
agaccatcgtgagataagaaaaaaaaaatatgacagaactagacactctt

SEQ ID NO: 8 - DNA, 50 bp, synthetic construct
  source 1..50: mol_type = other DNA
ctcctggcatcagctaagaaaaaaaaaatatgacagaactagacactctt

SEQ ID NO: 9 - DNA, 50 bp, synthetic construct
  source 1..50: mol_type = other DNA
gggagacaatgttaaatcaaaaaaaaaatatgacagaactagacactctt

SEQ ID NO: 10 - DNA, 49 bp, synthetic construct
  source 1..49: mol_type = other DNA
aaaacagcaagctagtcaaaaaaaaaatatgacagaactagacactctt

SEQ ID NO: 11 - DNA, 32 bp, synthetic construct
  source 1..32: mol_type = other DNA
aaaaaaaaaatatgacagaactagacactctt

SEQ ID NO: 12 - DNA, 22 bp, synthetic construct
  source 1..22: mol_type = other DNA
tatgacagaactagacactctt

SEQ ID NO: 13 - DNA, 114 bp, synthetic construct
  source 1..114: mol_type = other DNA
  misc_feature 112..114: note = RNA
  misc_feature 1..111: note = DNA
tagggaattcgtcgacggatcccgtggcgtctgcaacggaaaagaatttatcttgtcctgcaggtcgacgcatgcgccgaaaaaaaaaagacgctaatagttaagacgcttttt

SEQ ID NO: 14 - DNA, 35 bp, synthetic construct
  source 1..35: mol_type = other DNA
  misc_feature 33..35: note = RNA
  misc_feature 1..32: note = DNA
aaaaaaaaaagacgctaatagttaagacgcttttt

SEQ ID NO: 15 - DNA, 22 bp, synthetic construct
  source 1..22: mol_type = other DNA
gacgctaatagttaagacgctt

SEQ ID NO: 16 - DNA, 114 bp, synthetic construct
  source 1..114: mol_type = other DNA
  misc_feature 112..114: note = RNA
  misc_feature 1..111: note = DNA
cggcgcatgcgtcgacctgcaggacaagataaattcttttccgttgcagacgccacgggatccgtcgacgaattccctaaaaaaaaaaagacgctaatagttaagacgcttttt

SEQ ID NO: 17 - DNA, 65 bp, synthetic construct
  source 1..65: mol_type = other DNA
  modified_base 1: mod_base = OTHER; note = phosphate
ctattagcgtccagtgaatgcgagtccgtctaagagagtagtacagcagccgtcaagagtgtcta

SEQ ID NO: 18 - DNA, 25 bp, synthetic construct
  source 1..25: mol_type = other DNA
  modified_base 1: mod_base = OTHER; note = Phosphate
gttctgtcatatttaagcgtcttaa

SEQ ID NO: 19 - DNA, 23 bp, synthetic construct
  source 1..23: mol_type = other DNA
ttttttttttttttttttttttt

SEQ ID NO: 20 - DNA, 20 bp, synthetic construct
  source 1..20: mol_type = other DNA
  unsure 1: note = Alexa 647 linked
cagtgaatgcgagtccgtct

SEQ ID NO: 21 - DNA, 22 bp, synthetic construct
  source 1..22: mol_type = other DNA
ctgacatactgtcctagcgatt

SEQ ID NO: 22 - DNA, 18 bp, synthetic construct
  source 1..18: mol_type = other DNA
cagtatgtcagcccagag

SEQ ID NO: 23 - DNA, 69 bp, synthetic construct
  source 1..69: mol_type = other DNA
cgtccaaatgattttcagtgaatgcgagtccgtctaagagagtagtacagcagccgtcaatcgctagga

SEQ ID NO: 24 - DNA, 22 bp, synthetic construct
  source 1..22: mol_type = other DNA
  unsure 1: note = 5Cy5 label
aatcgctaggacagtatgtcag

SEQ ID NO: 25 - DNA, 22 bp, synthetic construct
  source 1..22: mol_type = other DNA
ccgaataggtgctttgaagcac

SEQ ID NO: 26 - DNA, 22 bp, synthetic construct
  source 1..22: mol_type = other DNA
ccgaataggtgctttgaagcac

SEQ ID NO: 27 - DNA, 69 bp, synthetic construct
  source 1..69: mol_type = other DNA
cgtccaaatgattttcagtgaatgcgagtccgtctaagagagtagtacagcagccgtcgtgcttcaaag

SEQ ID NO: 28 - DNA, 22 bp, synthetic construct
  source 1..22: mol_type = other DNA
  unsure 1: note = 36-TAMTSp label
gtgcttcaaagcacctattcgg

SEQ ID NO: 29 - DNA, 22 bp, synthetic construct
  source 1..22: mol_type = other DNA
gcccttgttaagaccatttgtg

SEQ ID NO: 30 - DNA, 18 bp, synthetic construct
  source 1..18: mol_type = other DNA
ttaacaagggccccagag

SEQ ID NO: 31 - DNA, 69 bp, synthetic construct
  source 1..69: mol_type = other DNA
cgtccaaatgattttcagtgaatgcgagtccgtctaagagagtagtacagcagccgtccacaaatggtc

SEQ ID NO: 32 - DNA, 22 bp, synthetic construct
  source 1..22: mol_type = other DNA
  unsure 1: note = 56-FAM label
cacaaatggtcttaacaagggc

SEQ ID NO: 33 - DNA, 110 bp, synthetic construct
  source 1..110: mol_type = other DNA
  misc_feature 108..110: note = RNA
  misc_feature 1..107: note = DNA
tagggaattcgtcgacggatcccgtggcgtctgcaacggaaaagaatttatcttgtcctgcaggtcgacgcatgcgccgaaaaaaaaaatcatttggacgctctgggttt

SEQ ID NO: 34 - DNA, 82 bp, synthetic construct
  source 1..82: mol_type = other DNA
ctgggaaaaccaccttcgtgatcatggtatctcccctgccaggtaagtataaaaaaaaaactgacatactgtcctagcgatt

SEQ ID NO: 35 - DNA, 82 bp, synthetic construct
  source 1..82: mol_type = other DNA
cgaacgcagtcccccactaccacaaattatgcagtcgagtttcccacattaaaaaaaaaactgacatactgtcctagcgatt

SEQ ID NO: 36 - DNA, 50 bp, synthetic construct
  source 1..50: mol_type = other DNA
agaccatcgtgagataagaaaaaaaaaaccgaataggtgctttgaagcac

SEQ ID NO: 37 - DNA, 50 bp, synthetic construct
  source 1..50: mol_type = other DNA
ctcctggcatcagctaagaaaaaaaaaaccgaataggtgctttgaagcac

SEQ ID NO: 38 - DNA, 50 bp, synthetic construct
  source 1..50: mol_type = other DNA
gggagacaatgttaaatcaaaaaaaaaagcccttgttaagaccatttgtg

SEQ ID NO: 39 - DNA, 49 bp, synthetic construct
  source 1..49: mol_type = other DNA
aaaacagcaagctagtcaaaaaaaaaagcccttgttaagaccatttgtg

(19) United States
(12) Patent Application Publication
(10) Pub. No.: US 2026/0348949 A1
(43) Pub. Date: Nov. 5, 2026

(54) Compositions and Methods for GlycoRNA Imaging via Proximity Ligation Assay

(71) Applicant: Board of Regents, The University of Texas System, Austin, TX (US)

(72) Inventors: Yi Lu, Austin, TX (US); Yuan Ma, Austin, TX (US); Weijie Guo, Austin, TX (US); Quanbing Mou, Austin, TX (US)

(21) Appl. No.: 19/104,918

(22) PCT Filed: Aug. 25, 2023
(86) PCT No.: PCT/US2023/072898

§ 371 Date: Feb. 19, 2025

Related U.S. Application Data
(60) Provisional application No. 63/373,605, filed on Aug. 26, 2022.

(51) Int. Cl. G01N 33/53; C12Q 1/682

(52) U.S. Cl. CPC G01N 33/5308; C12Q 1/682; G01N 2440/38

(57) ABSTRACT

The present invention describes compositions for use in a proximity assay for detecting glycosylated molecules in a sample, and methods of use of the compositions for a proximity assay for detecting glycosylated molecules.

### CROSS REFERENCE TO RELATED APPLICATIONS

This application claims priority to U.S. Provisional Application No. 63/373,605, filed Aug. 26, 2022, which is hereby incorporated by reference herein in its entirety.

### STATEMENT REGARDING FEDERALLY SPONSORED RESEARCH OR DEVELOPMENT

This invention was made with government support under Grant no. R35 GM141931 awarded by the National Institutes of Health. The government has certain rights in the invention.

### BACKGROUND OF THE INVENTION

Cellular glycans play important roles in biology by regulating many essential functions, including cell communications, homeostasis, immunomodulation, and embryogenesis (Reily, C., et al., 2019, Nature Reviews Nephrology, 15:346-366). While proteoglycans, glycoproteins, and glycosphingolipids have been widely studied, glycosylated RNAs (glycoRNAs) have recently been discovered on the surfaces of multiple cell types, organisms, and species (Flynn, R. A., et al., 2021, Cell, 184:3109-3124). RNA is a major class of biomacromolecules with various modifications (e.g., pseudo-uridylation, methylation, and adenylation) that contribute to diverse cellular processes including tRNA-mediated translation, epigenetics, chromatin structure modulation, and RNA maturation (Suzuki, T., 2021, Nature Reviews Molecular Cell Biology, 22:375-392; Tyagi, W., et al., 2022, Cancer Gene Therapy; Helm, M., et al., 2017, Nature Reviews Genetics, 18:275-291; Li, Y., et al., 2020, Nature Genetics, 52:870-877; Yu, S., et al., 2020, Nature Reviews Molecular Cell Biology, 21:542-556). Despite the discovery of this new RNA modification, the detailed information on the glycoRNAs, especially their spatial distributions and expression levels on the surface of living cells, remains to be understood. Such information is critical to understand the biological functions of glycoRNAs and their roles in different diseases.

Several methods have been reported to identify and measure glycoRNAs, but most of them study only extracted glycoRNAs in test tubes (Flynn, R. A., et al., 2021, Cell, 184:3109-3124). For example, RNA blot can analyze biotinylated glycoRNAs labeled by bioorthogonal reactions and compare their relative abundance between different samples. However, it can detect only the overall glycoRNAs but cannot differentiate individual glycoRNA with sequence specificity. To address this issue, next-generation sequencing (NGS) is utilized to obtain the RNA sequences of biotinylated glycoRNA isolated by streptavidin magnetic beads (Flynn, R. A., et al., 2021, Cell, 184:3109-3124). To study the glycan moiety of glycoRNA, high-performance liquid chromatography (HPLC) and mass spectrometry (MS) can reveal glycan composition and conformation, but cannot directly visualize glycoRNAs on cell surfaces (Flynn, R. A., et al., 2021, Cell, 184:3109-3124). In addition, anti-dsRNA antibodies or glycan receptors (e.g., siglec, lectin) have been used to verify the RNA or glycan on cell surfaces, respectively (Flynn, R. A., et al., 2021, Cell, 184:3109-3124). However, the anti-dsRNA antibodies cannot differentiate glycoRNAs with other surface RNAs and cannot distinguish RNAs with different sequences, even if the RNA sequences are known to be critical for the RNA functions (Huang, H., et al., 2020, Genome Biology, 21:225). In addition, the glycan receptors are insufficient to identify glycoRNAs from other surface glycans in glycoproteins or glycolipids.

Therefore, there is a pressing need to develop a method to directly visualize glycosylated molecules in situ with high selectivity against other nucleic acid molecules and glycans on the cell surfaces, and also provide both sequence and spatial information to better understand the properties and biological functions of glycosylated molecules.

### SUMMARY OF THE INVENTION

In one embodiment, the invention relates to an assay system for detecting at least one glycosylated molecule, the system comprising:
a) a glycan binding molecule comprising a glycan binding domain, a spacer and a linker domain, wherein the linker domain comprises a first nucleotide sequence specific for binding to a first connector DNA molecule, referred to as “Connector A,” located 5′ and adjacent to a second nucleotide sequence specific for binding to a second connector DNA molecule, referred to as “Connector B;”
b) a target binding molecule comprising a target binding domain specific for binding to a target glycosylated molecule, a spacer and a linker domain, wherein the linker domain comprises a first nucleotide sequence specific for binding to Connector B located 5′ and adjacent to a second nucleotide sequence specific for binding to Connector A;
c) Connector A comprising a phosphorylated 5′ end specific for binding to the linker domain of the glycan binding molecule and a 3′ end specific for binding to the linker domain of the target binding molecule; and
d) Connector B comprising a phosphorylated 5′ end specific for binding to the linker domain of the target binding molecule and a 3′ end specific for binding to the linker domain of the glycan binding molecule.

In one embodiment, the system further comprises a labeled detection molecule.

In one embodiment, the target glycosylated molecule a glycoRNA molecule, a glycoDNA molecule, or a glycopeptide.

In one embodiment, the glycan binding domain is a protein, a peptide, a DNA, an RNA, an aptamer, a Siglec, a lectin, antibody, or a chemical ligand.

In one embodiment, the glycan-specific binding domain specifically binds to a sialic acid or sugar moiety. In one embodiment, the sialic acid or sugar moiety is N-acetylneuraminic acid (Neu5 Ac), N-Glycolylneuraminic acid (Neu5Gc), galactose, glucose or mannose. In one embodiment, the glycan binding domain comprises an aptamer specific for binding to Neu5Ac. In one embodiment, the glycan binding domain comprises the sequence as set forth in SEQ ID NO:1.

In one embodiment, at least one of the glycan-specific binding molecule, the target binding molecule, Connector A and Connector B comprises a detectable moiety. In one embodiment, at least one of the glycan-specific binding molecule, the target binding molecule, Connector A and Connector B comprises an emission donor molecule and at least one of the glycan-specific binding molecule, the target binding molecule, Connector A and Connector B comprises an emission acceptor molecule.

In one embodiment, the system comprises a combination of a glycan binding molecule comprising a glycan binding domain comprising SEQ ID NO:1 and a linker domain as set forth in SEQ ID NO:12, a target binding molecule comprising a linker domain as set forth in SEQ ID NO:15, Connector A comprising a sequence as set forth in SEQ ID NO:17, and Connector B comprising a sequence as set forth in SEQ ID NO: 18. In one embodiment, the system further comprises a labeled detection molecule comprising a sequence as set forth in SEQ ID NO:20.

In one embodiment, the system comprises a combination of a glycan binding molecule comprising SEQ ID NO:33, a target binding molecule comprising a linker domain as set forth in SEQ ID NO:21, Connector A comprising a sequence as set forth in SEQ ID NO:22, and Connector B comprising a sequence as set forth in SEQ ID NO: 23. In one embodiment, the system further comprises a labeled detection molecule comprising a sequence as set forth in SEQ ID NO:24.

In one embodiment, the system comprises a combination of a glycan binding molecule comprising SEQ ID NO:33, a target binding molecule comprising a linker domain as set forth in SEQ ID NO:25, Connector A comprising a sequence as set forth in SEQ ID NO:26, and Connector B comprising a sequence as set forth in SEQ ID NO: 27. In one embodiment, the system further comprises a labeled detection molecule comprising a sequence as set forth in SEQ ID NO:28.

In one embodiment, the system comprises a combination of a glycan binding molecule comprising SEQ ID NO:33, a target binding molecule comprising a linker domain as set forth in SEQ ID NO:29, Connector A comprising a sequence as set forth in SEQ ID NO:30, and Connector B comprising a sequence as set forth in SEQ ID NO: 31. In one embodiment, the system further comprises a labeled detection molecule comprising a sequence as set forth in SEQ ID NO:32.

In one embodiment, the invention relates to an assay for detecting at least one glycosylated molecule, the method comprising:
a) contacting a sample with:
   i) a glycan binding molecule comprising a glycan binding domain, a spacer and a linker domain, wherein the linker domain comprises a first nucleotide sequence specific for binding to a first connector DNA molecule, referred to as “Connector A,” located 5′ and adjacent to a second nucleotide sequence specific for binding to a second connector DNA molecule, referred to as “Connector B;”
   ii) a target binding molecule comprising a target binding domain specific for binding to a target glycosylated molecule, a spacer and a linker domain, wherein the linker domain comprises a first nucleotide sequence specific for binding to Connector B located 5′ and adjacent to a second nucleotide sequence specific for binding to Connector A;
   iii) Connector A comprising a phosphorylated 5′ end specific for binding to the linker domain of the glycan binding molecule and a 3′ end specific for binding to the linker domain of the target binding molecule; and
   iv) Connector B comprising a phosphorylated 5′ end specific for binding to the linker domain of the target binding molecule and a 3′ end specific for binding to the linker domain of the glycan binding molecule; and
b) detecting the proximity of the Connector A and Connector B molecules.

In one embodiment, the glycosylated molecule is selected from the group consisting of a glycoRNA molecule, a glycoDNA molecule, and a glycopeptide.

In one embodiment, the glycan binding domain is a protein, a peptide, a DNA, an RNA, an aptamer, a Siglec, a lectin, antibody, or a chemical ligand.

In one embodiment, the glycan-specific binding domain specifically binds to a sialic acid or sugar moiety.

In one embodiment, the sialic acid or sugar moiety is N-acetylneuraminic acid (Neu5Ac), N-Glycolylneuraminic acid (Neu5Gc), galactose, glucose or mannose.

In one embodiment, the glycan binding domain comprises an aptamer specific for binding to Neu5 Ac.

In one embodiment, the method of detecting the proximity of the Connector A and Connector B molecules is ligation followed by rolling circle amplification and probe based detection, ligation followed by PCR based amplification and detection, FRET, Flow cytometry, ligation followed by hybridization chain reaction (HCR), ligation followed by catalytic hairpin assembly (CHA), or ligation followed by detection using DNA-PAINT probes.

In one embodiment, at least one of the glycan-specific binding molecule, the target binding molecule, Connector A and Connector B comprises a detectable moiety. In one embodiment, at least one of the glycan-specific binding molecule, the target binding molecule, Connector A and Connector B comprises an emission donor molecule and at least one of the glycan-specific binding molecule, the target binding molecule, Connector A and Connector B comprises an emission acceptor molecule.

In one embodiment, the method comprises:
a) contacting a sample with:
   i) the glycan binding molecule comprising the glycan binding domain, the spacer and the linker domain, wherein the linker domain comprises a first nucleotide sequence specific for binding to Connector A, located 5′ and adjacent to a second nucleotide sequence specific for binding to Connector B;
   ii) the target binding molecule comprising the target binding domain specific for binding to a target glycosylated molecule, the spacer and the linker domain, wherein the linker domain comprises a first nucleotide sequence specific for binding to Connector B located 5′ and adjacent to a second nucleotide sequence specific for binding to Connector A;
   iii) Connector A comprising a phosphorylated 5′ end specific for binding to the linker domain of the glycan binding molecule and a 3′ end specific for binding to the linker domain of the target binding molecule; and
   iv) Connector B comprising a phosphorylated 5′ end specific for binding to the linker domain of the target binding molecule and a 3′ end specific for binding to the linker domain of the glycan binding molecule; and
   v) a DNA ligase to ligate the Connector A and Connector B; and
b) contacting the sample with a polymerase for rolling circle amplification of the ligated connector DNA product;
c) contacting the sample with a labeled detection molecule specific for binding to a junction formed from ligation of Connector A and Connector B; and
d) detecting the labeled detection molecule.

In one embodiment, the glycosylated molecule is a glycoRNA molecule, a glycoDNA molecule, and a glycopeptide.

In one embodiment, the glycan binding domain is a protein, a peptide, a DNA, an RNA, an aptamer, a Siglec, a lectin, antibody, or a chemical ligand.

In one embodiment, the glycan-specific binding domain specifically binds to a sialic acid or sugar moiety.

In one embodiment, the sialic acid or sugar moiety is N-acetylneuraminic acid (Neu5Ac), N-Glycolylneuraminic acid (Neu5Gc), galactose, glucose and mannose. In one embodiment, the glycan binding domain comprises an aptamer specific for binding to Neu5Ac.

In one embodiment, the method of detecting the labeled detection molecule is fluorescence detection methods, Flow cytometry, hybridization chain reaction (HCR), catalytic hairpin assembly (CHA), or using DNA-PAINT probes.

In one embodiment, the method comprises a combination of a glycan binding molecule comprising a glycan binding domain comprising SEQ ID NO:1 and a linker domain as set forth in SEQ ID NO:12, a target binding molecule comprising a linker domain as set forth in SEQ ID NO: 15, Connector A comprising a sequence as set forth in SEQ ID NO: 17, Connector B comprising a sequence as set forth in SEQ ID NO:18, and a labeled detection molecule comprising a sequence as set forth in SEQ ID NO:20.

In one embodiment, the method comprises a combination of a glycan binding molecule comprising a sequence as set forth in SEQ ID NO:33, a target binding molecule comprising a linker domain as set forth in SEQ ID NO:21, Connector A comprising a sequence as set forth in SEQ ID NO:22, Connector B comprising a sequence as set forth in SEQ ID NO:23, and a labeled detection molecule comprising a sequence as set forth in SEQ ID NO:24.

In one embodiment, the method comprises a combination of a glycan binding molecule comprising a sequence as set forth in SEQ ID NO:33, a target binding molecule comprising a linker domain as set forth in SEQ ID NO:25, Connector A comprising a sequence as set forth in SEQ ID NO:26, Connector B comprising a sequence as set forth in SEQ ID NO:27, and a labeled detection molecule comprising a sequence as set forth in SEQ ID NO:28.

In one embodiment, the method comprises a combination of a glycan binding molecule comprising a sequence as set forth in SEQ ID NO:33, a target binding molecule comprising a linker domain as set forth in SEQ ID NO:29, Connector A comprising a sequence as set forth in SEQ ID NO:30, Connector B comprising a sequence as set forth in SEQ ID NO:31, and a labeled detection molecule comprising a sequence as set forth in SEQ ID NO:32.

In one embodiment, the DNA ligase comprises T4 DNA ligase.

In one embodiment, the invention relates to a multiplex assay for detection of at least two target glycosylated molecules in a sample. In one embodiment, the multiplex assay comprises at least two of:
a) a glycan binding molecule comprising a glycan binding domain comprising SEQ ID NO: 1 and a linker domain as set forth in SEQ ID NO:12, a target binding molecule comprising a linker domain as set forth in SEQ ID NO:15, Connector A comprising a sequence as set forth in SEQ ID NO:17, Connector B comprising a sequence as set forth in SEQ ID NO:18, and a labeled detection molecule comprising a sequence as set forth in SEQ ID NO:20;
b) a glycan binding molecule comprising a sequence as set forth in SEQ ID NO:33, a target binding molecule comprising a linker domain as set forth in SEQ ID NO:21, Connector A comprising a sequence as set forth in SEQ ID NO:22, Connector B comprising a sequence as set forth in SEQ ID NO:23, and a labeled detection molecule comprising a sequence as set forth in SEQ ID NO:24;
c) a glycan binding molecule comprising a sequence as set forth in SEQ ID NO:33, a target binding molecule comprising a linker domain as set forth in SEQ ID NO:25, Connector A comprising a sequence as set forth in SEQ ID NO:26, Connector B comprising a sequence as set forth in SEQ ID NO:27, and a labeled detection molecule comprising a sequence as set forth in SEQ ID NO:28; and
d) a glycan binding molecule comprising a sequence as set forth in SEQ ID NO:33, a target binding molecule comprising a linker domain as set forth in SEQ ID NO:29, Connector A comprising a sequence as set forth in SEQ ID NO:30, Connector B comprising a sequence as set forth in SEQ ID NO:31, and a labeled detection molecule comprising a sequence as set forth in SEQ ID NO:32.

In one embodiment, the sample is a biological sample or a patient sample.

In one embodiment, the assay is for use in diagnosing a disease or disorder in the subject, monitoring the progression of a disease or disorder in the subject providing a disease prognosis, or evaluating the effects of a treatment provided to a subject.

In one embodiment, the disease or disorder is an inflammatory disease or disorder or cancer.

In one embodiment, the invention relates to a kit comprising a combination of components for performing an assay or multiplex assay for detection of at least one glycosylated molecule.

In one embodiment, the invention relates to a method of diagnosing breast cancer in a subject in need thereof, the method comprising
a) obtaining a sample from a patient;
b) contacting the sample with
   i) a glycan binding molecule comprising a glycan binding domain, a spacer and a linker domain, wherein the linker domain comprises a first nucleotide sequence specific for binding to a first connector DNA molecule, referred to as “Connector A,” located 5′ and adjacent to a second nucleotide sequence specific for binding to a second connector DNA molecule, referred to as “Connector B;”
   ii) a target binding molecule comprising a target binding domain specific for binding to a target glycosylated molecule, a spacer and a linker domain, wherein the linker domain comprises a first nucleotide sequence specific for binding to Connector B located 5′ and adjacent to a second nucleotide sequence specific for binding to Connector A;
   iii) Connector A comprising a phosphorylated 5′ end specific for binding to the linker domain of the glycan binding molecule and a 3′ end specific for binding to the linker domain of the target binding molecule; and
   iv) Connector B comprising a phosphorylated 5′ end specific for binding to the linker domain of the target binding molecule and a 3′ end specific for binding to the linker domain of the glycan binding molecule; and
   v) a DNA ligase to ligate Connector A and Connector B;
c) contacting the sample with a polymerase for rolling circle amplification of the ligated connector DNA product;
d) contacting the sample with a labeled detection molecule specific for binding to a junction formed from ligation of Connector A and Connector B;
e) detecting the labeled detection molecule;
f) comparing the level of at least one glycosylated molecule detected in the patient sample to a comparator control level of the glycosylated molecule;
g) diagnosing the subject as having or at risk of breast cancer when a lower level of at least one glycosylated molecule is detected in the patient sample as compared to the control.

In one embodiment, at least one glycosylated molecule is U1 glycoRNA, U3 glycoRNA, U8 glycoRNA, U35a glycoRNA, or Y5 glycoRNA.

In one embodiment, the method comprises contacting the sample with at least one of:
a) a glycan binding molecule comprising SEQ ID NO: 13, a target binding molecule selected from the group consisting of SEQ ID NO:2-SEQ ID NO:10; Connector A comprising a sequence as set forth in SEQ ID NO:17, Connector B comprising a sequence as set forth in SEQ ID NO:18, and a labeled detection molecule comprising a sequence as set forth in SEQ ID NO:20;
b) a glycan binding molecule comprising a sequence as set forth in SEQ ID NO:33, a target binding molecule selected from the group consisting of SEQ ID NO:34 and SEQ ID NO: 35, Connector A comprising a sequence as set forth in SEQ ID NO:22, Connector B comprising a sequence as set forth in SEQ ID NO:23, and a labeled detection molecule comprising a sequence as set forth in SEQ ID NO:24;
c) a glycan binding molecule comprising a sequence as set forth in SEQ ID NO:33, a target binding molecule selected from the group consisting of SEQ ID NO:36 and SEQ ID NO: 37, Connector A comprising a sequence as set forth in SEQ ID NO:26, Connector B comprising a sequence as set forth in SEQ ID NO:27, and a labeled detection molecule comprising a sequence as set forth in SEQ ID NO:28; and
d) a glycan binding molecule comprising a sequence as set forth in SEQ ID NO:33, a target binding molecule selected from the group consisting of SEQ ID NO:38 and SEQ ID NO: 39, Connector A comprising a sequence as set forth in SEQ ID NO:30, Connector B comprising a sequence as set forth in SEQ ID NO:31, and a labeled detection molecule comprising a sequence as set forth in SEQ ID NO:32.

In one embodiment, the method further comprises administering a treatment for the diagnosed breast cancer.

In one embodiment, the invention relates to a method of determining the prognosis of breast cancer in a subject in need thereof, the method comprising
a) obtaining a sample from a subject diagnosed as having breast cancer;
b) contacting the sample with
   i) a glycan binding molecule comprising a glycan binding domain, a spacer and a linker domain, wherein the linker domain comprises a first nucleotide sequence specific for binding to a first connector DNA molecule, referred to as “Connector A,” located 5′ and adjacent to a second nucleotide sequence specific for binding to a second connector DNA molecule, referred to as “Connector B;”
   ii) a target binding molecule comprising a target binding domain specific for binding to a target glycosylated molecule, a spacer and a linker domain, wherein the linker domain comprises a first nucleotide sequence specific for binding to Connector B located 5′ and adjacent to a second nucleotide sequence specific for binding to Connector A;
   iii) Connector A comprising a phosphorylated 5′ end specific for binding to the linker domain of the glycan binding molecule and a 3′ end specific for binding to the linker domain of the target binding molecule; and
   iv) Connector B comprising a phosphorylated 5′ end specific for binding to the linker domain of the target binding molecule and a 3′ end specific for binding to the linker domain of the glycan binding molecule; and
   v) a DNA ligase to ligate Connector A and Connector B;
c) contacting the sample with a polymerase for rolling circle amplification of the ligated connector DNA product;
d) contacting the sample with a labeled detection molecule specific for binding to a junction formed from ligation of Connector A and Connector B;
e) detecting the labeled detection molecule;
f) comparing the level of at least one glycosylated molecule detected in the patient sample to a comparator control level of the glycosylated molecule;
g) identifying the subject as having a poorer prognosis for the diagnosed breast cancer when a lower level of at least one glycosylated molecule is detected in the patient sample as compared to the control.

In one embodiment, at least one glycosylated molecule is U1 glycoRNA, U3 glycoRNA, U8 glycoRNA, U35a glycoRNA, or Y5 glycoRNA.

In one embodiment, the method comprises contacting the sample with at least one of:
a) a glycan binding molecule comprising SEQ ID NO:13, a target binding molecule selected from the group consisting of SEQ ID NO:2-SEQ ID NO:10; Connector A comprising a sequence as set forth in SEQ ID NO:17, Connector B comprising a sequence as set forth in SEQ ID NO:18, and a labeled detection molecule comprising a sequence as set forth in SEQ ID NO:20;
b) a glycan binding molecule comprising a sequence as set forth in SEQ ID NO:33, a target binding molecule selected from the group consisting of SEQ ID NO:34 and SEQ ID NO: 35, Connector A comprising a sequence as set forth in SEQ ID NO:22, Connector B comprising a sequence as set forth in SEQ ID NO:23, and a labeled detection molecule comprising a sequence as set forth in SEQ ID NO:24;
c) a glycan binding molecule comprising a sequence as set forth in SEQ ID NO:33, a target binding molecule selected from the group consisting of SEQ ID NO:36 and SEQ ID NO: 37, Connector A comprising a sequence as set forth in SEQ ID NO:26, Connector B comprising a sequence as set forth in SEQ ID NO:27, and a labeled detection molecule comprising a sequence as set forth in SEQ ID NO:28; and
d) a glycan binding molecule comprising a sequence as set forth in SEQ ID NO:33, a target binding molecule selected from the group consisting of SEQ ID NO:38 and SEQ ID NO: 39, Connector A comprising a sequence as set forth in SEQ ID NO:30, Connector B comprising a sequence as set forth in SEQ ID NO:31, and a labeled detection molecule comprising a sequence as set forth in SEQ ID NO:32.

In one embodiment, the method further comprises administering a treatment based on the prognostic outcome for the diagnosed breast cancer.

### BRIEF DESCRIPTION OF THE DRAWINGS

The following detailed description of various embodiments of the invention will be better understood when read in conjunction with the appended drawings. For the purpose of illustrating the invention, there are shown in the drawings, illustrative embodiments. It should be understood, however, that the invention is not limited to the precise arrangements and instrumentalities of the embodiments shown in the drawings.

FIG. 1A and FIG. 1B depict a schematic illustration of glycoRNA in situ imaging utilizing Sialic Acid Aptamer and RNA in situ Hybridization-mediated Proximity Ligation Assay (ARPLA). FIG. 1A depicts a representative schematic of the structure and functional domains of a glycan probe, RNA binding probe, and connectors. FIG. 1B depicts a schematic detailing the process of ARPLA for glycoRNA recognition and signal amplification.

FIG. 2A through FIG. 2E depict representative verifications of ARPLA using HeLa as a model cell line. FIG. 2A depicts a representative thermogram for the ITC titration of 20 μM Neu5 Ac aptamer titrated by 1 mM Neu5Ac in aptamer binding buffer. FIG. 2B depicts integrated heat of the ITC titration for Neu5Ac aptamer and Neu5Ac, the black line represents the binding curve fitted with the “one set of binding sites” model. FIG. 2C depicts representative imaging of blotting for total RNA from HeLa cells after metabolic labeling with Ac₄ManNAz, or HeLa cells without metabolic labeling. FIG. 2D depicts representative ARPLA-mediated glycoRNA imaging on the surface of HeLa, HL-60, and THP-1 cells. Scale bar: 20 μm (HeLa), 10 μm (HL-60, THP-1). FIG. 2E depicts representative transmission-through dye microscopic images of HeLa cells. The membrane permeable dye (CellTracker Orange CMRA) and membrane impermeable quencher acid blue 9 (AB9) were applied to the same cells. AB9 cannot enter a cell with an intact membrane and thus cannot quench the membrane permeable dye, so only cells with intact membranes show bright fluorescence signals from CellTracker Orange CMRA. Leaky or damaged membranes after permeabilization treatment allows the quencher to enter the cells, resulting in reduced or diminished fluorescence. Scale bar: 40 μm.

FIG. 3A through FIG. 3D depict representative results for the evaluation of ARPLA for glycoRNA imaging in HeLa cells. FIG. 3A depicts a schematic representation of the ARPLA and a variety of control groups in the absence of all components for ARPLA and representative confocal laser scanning microscopy (CLSM) imaging in HeLa cells for each condition. FIG. 3B depicts quantification of the average fluorescence intensity (a.u.) per cell in FIG. 3A. FIG. 3C depicts representative imaging of validation of ARPLA specificity. The HeLa cells imaged were pretreated with RNase, glycosylation inhibitors, or endoglycosidases, respectively, and analyzed by ARPLA. FIG. 3D depicts quantification of the average fluorescence intensity per cell in FIG. 3C. Data (FIGS. 3B and 3D) are representative of three independent experiments, n=5 frames. Data are presented as mean±S.D. Statistical significance was determined using an unpaired two-tailed Student's t-test; ns, not significant; *, p<0.05; **; p<0.01; ***, p<0.001. Scale bars for FIGS. 2A and 2B: 40 μm.

FIG. 4A through FIG. 4E depict representative evaluation of the generality of ARPLA. FIG. 4A depicts representative visualization of glycoRNA with various RNA sequences, including U3, U8, U35a, and Y5. FIG. 4B depicts representative CLSM images of U1 glycoRNA in different cell lines, such as SH-SY5Y, PANC-1, and HEK293T. FIG. 4C depicts representative blotting of total RNA from SH-SY5Y cells with or without metabolic labeling with Ac4ManNAz. FIG. 4D depicts representative blotting of total RNA from PANC-1 cells with or without metabolic labeling with Ac4ManNAz. FIG. 4E depicts representative blotting of total RNA from HEK293T cells with or without metabolic labeling with Ac4ManNAz. Scale bars: 40 μm.

FIG. 5A and FIG. 5B depict representative distributions of glycoRNAs as revealed by ARPLA in HL-60 cells. FIG. 5A depicts representative fluorescent imaging of lipid rafts, indicated by Alexa Fluor 555-labeled cholera toxin subunit B (top) or BODIPY dye-labeled sphingolipids (bottom), and glycoRNA, represented by ARPLA with a U1 probe. The overlay images and plot profiles display the colocalization between glycoRNA and lipid rafts. Pearson's coefficient r=0.67 (CT-B) and r=0.46 (BODIPY). FIG. 5B depicts representative immunofluorescent images of t-SNARE (top) and v-SNARE (bottom) with glycoRNA indicating the intracellular trafficking of glycoRNAs. The overlay images and plot profiles showed a colocalization between glycol RNAs and t-SNARE (Pearson's coefficient r=0.70) or v-SNARE (Pearson's coefficient r=0.58). For FIGS. 5A and 5B, scale bars: 5 μm. The intensity of different fluorochromes along the line in the overlay images are shown in the histograms on the right.

FIG. 6A through FIG. 6C depict representative visualizations of glycoRNA in malignant transformation using ARPLA (see FIG. 7). FIG. 6A depicts representative CLSM images of MCF-10A, MCF-7, and MDA-MB-231 cells in control groups using scrambled DNA with U1 and U35 glycoRNA. FIG. 6B depicts representative CLSM images of MCF-10A, MCF-7, and MDA-MB-231 cells in control groups using scrambled DNA with Y5 glycoRNA. FIG. 6C depicts a representative agarose gel electrophoresis image of total RNA from MCF-10A, MCF-7, and MDA-MB-231 cells treated with Ac₄ManNAz for 72 hours before RNA extraction.

FIG. 7A through FIG. 7C depicts representative visualizations of glycoRNAs in malignant transformation using ARPLA. FIG. 7A depicts representative CLSM imaging of U1, U35a, and 75 glycoRNA by ARPLA in MCF-10A, MCF-7, and MDA-MB-231 cells. Scale bar: 40 μm. FIG. 7B depicts a heatmap and quantitative analysis of relative fluorescence intensity in FIG. 7A (see also FIGS. 6A and 5B), n=3 biological replicates. FIG. 7C depicts representative imaging of total glycoRNA expression levels in MCF-10A, MCF-7, and MDA-MB-231 cells as evaluated by RNA blot. GlycoRNAs were metabolically labeled by AcManNAz.

FIG. 8A through FIG. 8F depict representative visualizations and quantification of glycoRNA level during THP-1 differentiation and LPS activation. FIG. 8A depicts a schematic representation of the process for differentiation and activation of THP-1 cells. FIG. 8B depicts representative CLSM images of U1, U35a, and Y5 glycoRNA levels investigated by ARPLA in THP-1 monocytes, resting M0 macrophages, and M0 macrophages activated by LPS. Scale bar: 40 μm. FIG. 8C depicts quantification of fluorescence intensity per cell in FIGS. 8B, 9A, and 9B, n=5 frames. FIG. 8D depicts a representative image of an RNA blot for total glycoRNA levels in THP-1 monocytes, resting M0 macrophages, and M0 macrophages activated by LPS. FIG. 8E depicts quantification of representative cell attachment levels in THP-1 models after RNase treatment. FIG. 8F depicts quantification of representative cell attachment levels in HL-60 models after RNase treatment. Data in FIGS. 8E and 8F are normalized to their respective control groups. Data are representative of 3 independent experiments; n=6 technical replicates. Data are presented as mean±S.D. Statistical analysis was performed using an unpaired two-tailed Student's t-test; ns, not significant; *, p<0.05; **, p<0.01; **, p<0.001.

FIG. 9A through FIG. 9D depicts representative visualizations of glycoRNA levels during THP-1 differentiation and activation by LPS (see also FIG. 8). FIG. 9A depicts representative CLSM images of THP-1 monocytes, resting M0 macrophages, and M0 macrophages activated by LPS treated with scrambled sequences to replace the aptamer in ARPLA with U1 and U35 glycoRNA. FIG. 9B depicts representative CLSM images of THP-1 monocytes, resting M0 macrophages, and M0 macrophages activated by LPS treated with scrambled sequences to replace the aptamer in ARPLA with Y5 glycoRNA. FIG. 9C depicts representative blotting of total RNA from THP-1 cells with or without metabolic labeling with Ac₄ManNAz. FIG. 9D depicts a representative agarose gel electrophoresis image of total RNA from THP-1 monocytes, resting M0 macrophages, and M0 macrophages activated by LPS after being treated with Ac₄ManNAz for 72 hours before RNA extraction.

FIG. 10A through FIG. 10C depict representative measurements of glycoRNA levels during HL-60 differentiation. FIG. 10A depicts representative CLSM images of U1, U3, and U8 glycoRNA level evaluation by ARPLA in HL-60 and dHL-60 cells. FIG. 10B depicts representative CLSM images of U1, U3, and U8 glycoRNA level evaluation in HL-60 and dHL-60 cells using DNA with scrambled sequences. FIG. 10C depicts quantification of relative fluorescence intensity of ARPLA in FIGS. 10A and 10B. Scale bar: 20 μm. Data are representative of three independent experiments, n=5 frames. Data are presented as mean±S.D. Statistical analysis was performed using an unpaired two-tailed Student's t-test; ns, not significant; *, p<0.05; **, p<0.01; ***, p<0.001.

FIG. 11A and FIG. 11B depict representative measurements of total glycoRNA levels during HL-60 differentiation by RNA blotting. FIG. 11A depicts representative blotting of total RNA from HL-60 cells with or without metabolic labeling with Ac₄ManNAz. FIG. 11B depicts representative blotting of total RNA from HL-60 and dHL-60 cells with or without metabolic labeling with Ac₄ManNAz. Cells were treated with Ac₄ManNAz for 72 hours before DNA extraction.

FIG. 12 depicts representative quantification of average cell attachment levels in resting M0 macrophages, M0 macrophages activated by LPS, and activated M0 macrophages after RNase treatment. Data are representative of three independent experiments, n=6. Data are presented as mean±S.D. Statistical analysis was performed using an unpaired two-tailed Student's t-test; ns, not significant; *, p<0.05; **, p<0.01; ***, p<0.001.

FIG. 13A through FIG. 13C depict 3D visualization of the spatial distributions of U1 glycoRNA and CT-B in HL-60 cells. Z-stack images were collected with the staining of U1 glycoRNA by ARPLA and lipid raft by CT-B. FIG. 13A depicts the images in z-slices format. FIG. 13B depicts the images in orthographic projection format. FIG. 13C depicts the images in maximum intensity projection format. Scale bar: 2 μm. The z-stack colocalization was repeated independently three times with similar results.

FIG. 14A through FIG. 14C depict simultaneous imaging of U1 and Y5 glycoRNAs with multiplex ARPLA in HL-60s. U1 and Y5 glycoRNAs were stained by orthogonal design of ARPLA sets. Z-stack images were collected to show their special distribution. FIG. 14A depicts the images in z-slices format. FIG. 14B depicts the images in orthographic projection format. FIG. 14C depicts the images in maximum intensity projection format. Scale bar: 5 μm.

FIG. 15 depict glycoRNA (U1) distributions at tissue level in the mouse brain.

### DETAILED DESCRIPTION

This invention is based, in part, on the development of a novel Sialic Acid Aptamer and RNA in situ Hybridization-mediated Proximity Ligation Assay (ARPLA) which can be used to visualize glycoRNAs on cell surfaces. ARPLA is a proximity assay which utilizes a sialic acid aptamer and an in situ hybridization probe for dual recognition of the glycan and RNA portions of the glycoRNA, respectively, followed by a proximity ligation to generate an intact circular DNA, which then serves as a template for RCA to produce ssDNA. Detection of the ssDNA therefore corresponds to the presence of a specific glycoRNA species. The invention also provides methods for using ARPLA for bioimaging, diagnostics, sensors, and therapeutics.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “aptamer” as used herein refers to any ligand that binds to a target molecule and includes oligo nucleic acid or peptide molecules that bind to a specific target molecule. Aptamers generally comprise one or more loops of variable sequence which function to interact with their target molecule.

The terms “biomarker” and “marker” are used herein interchangeably. They refer to a substance that is a distinctive indicator of a biological process, biological event and/or pathologic condition, disease or disorder.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

The term “detecting” or “detection,” means assessing the presence, absence, quantity or amount of a given substance (e.g., a reporter molecule) within a sample, including the derivation of qualitative or quantitative levels of such substances.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, “fragment” is defined as at least a portion of the variable region of the immunoglobulin molecule which binds to its target, i.e. the antigen binding region. Some of the constant region of the immunoglobulin may be included.

As used herein, “fused” means to couple directly or indirectly one molecule with another by whatever means, e.g., by covalent bonding, by non-covalent bonding, by ionic bonding, or by non-ionic bonding. Covalent bonding includes bonding by various linkers such as thioether linkers or thioester linkers. Direct fusion involves one molecule attached to the molecule of interest. Indirect fusion involves one molecule attached to another molecule which in turn is attached directly or indirectly to the molecule of interest.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “label” or a “detectable moiety” is a composition detectable by spectroscopic, photochemical, biochemical, immunochemical, chemical, magnetic resonance imaging, or other physical means.

As used herein, “nucleic acid” is meant to include any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). The term “nucleic acid” typically refers to large polynucleotides.

As used herein, the terms “peptide,” “polypeptide,” and “protein” are used interchangeably, and refer to a compound comprising amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein, is meant a molecule, such as a probe or aptamer, which recognizes and binds to another molecule or feature, but does not substantially recognize or bind other molecules or features in a sample.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Proximity assays are designed to generate a detectable signal when two or more molecules are detected in close proximity to each other. Proximity assays are widely used in studying protein-protein interaction, however proximity assays for detection of glycoRNAs have not previously been reported. The invention is based, in part on the development of a novel proximity assay for detection of glycoRNA in which probe specific for detection of a target nucleic acid molecule is used in combination with a sialic acid or sugar binding molecule. A signal is generated when the glycan-specific binding molecule and the nucleic acid-specific binding probe are in close proximity. In various embodiments, the invention encompasses compositions for use in performing the Sialic Acid Aptamer and RNA in situ Hybridization-mediated Proximity Ligation Assay (ARPLA) of the invention, as well as methods of detecting glycoRNAs in a sample using ARPLA.

### Glycan-Specific Binding Molecule

In some embodiments, the proximity assay of the invention comprises a glycan-specific binding molecule comprising a) a glycan binding domain comprising a molecule specific for binding to a sialic acid or sugar; b) a spacer domain; and c) a nucleic acid domain specific for binding to two different nucleic acid molecules that, when ligated, serves as a template for rolling circle amplification. In some embodiments, the nucleic acid molecules that, when ligated, serves as a template for rolling circle amplification are referred to herein as connector nucleic acid molecules. In some embodiments, the domain of the glycan-specific binding molecule comprising the nucleotide sequence specific for binding to the connector nucleic acid molecules is referred to herein as the linker domain. In some embodiments, the spacer domain is between the glycan binding domain and the linker domain.

In some embodiments, the glycan-specific binding molecule may be a protein, peptide, DNA, RNA, or a chimeric molecule such as an RNA/DNA molecule or a peptide/nucleic acid fusion molecule.

In some embodiments, the glycan binding domain may be a protein, peptide, DNA, RNA, Siglec, lectin, antibody, or a chemical ligand (e.g., DBCO for azide-labeled glycan and streptavidin for biotinlylated glycan). In some embodiments, the glycan-specific binding domain specifically binds to a sialic acid or sugar moiety. Exemplary glycan or sugar moieties include, but are not limited to, a sialic acid (e.g., N-acetylneuraminic acid (Neu5Ac) or N-Glycolylneuraminic acid (Neu5Gc)), galactose, glucose and mannose which are found in glycoRNA molecules. In some embodiments, the glycan or sugar binding domain is specific for binding to Neu5Ac. In some embodiments the glycan-specific binding domain is an aptamer comprising a DNA sequence that forms a 3-Dimensional structure that specifically binds to a sialic acid or sugar moiety. In some embodiments the glycan-specific binding domain is a Neu5Ac specific aptamer comprising the sequence:

(SEQ ID NO: 1)
TAGGGAATTCGTCGACGGATCCCGTGGCGTCTGCAACGGA
AAAGAATTTATCTTGTCCTGCAGGTCGACGCATGCGCCG.

The spacer in the glycan-specific binding molecule is generally between the glycan binding domain and the linker domain. In some embodiments, the spacer domain moves the linker domain away from the glycan-binding domain to enable the linker sequence to come into contact with the connector sequences and subsequent ligation of the connector sequences. In some embodiments, the spacer sequence comprises a flexible sequence 8-15 nucleotides long. In some embodiments, the spacer sequence comprises a poly A sequence 8-15 nucleotides long. In some embodiments, the spacer sequence comprises a poly A sequence 10 nucleotides long. The spacer length can be selected to optimize the proximity ligation distances.

In some embodiments, the linker domain of the glycan-specific binding molecule comprises a) a first nucleotide sequence specific for binding to a first connector molecule (referred to as “Connector A”), b) a second nucleotide sequence specific for binding to a second connector molecule (referred to as “Connector B”) and c) at least one nucleotide, modified nucleotide or moiety that protects or caps the 3′ end of the linker domain of the glycan specific binding molecule. In some embodiments, the first nucleotide sequence specific for binding to Connector A comprises 8-20 nucleotides specific for binding to the 5′ end of Connector A. In some embodiments, the second nucleotide sequence specific for binding to Connector B comprises 8-20 nucleotides specific for binding to the 3′ end of Connector B. In some embodiments, the 5′ end of Connector A and Connector B is phosphorylated. In some embodiments, the nucleotide sequence for binding to Connector A is directly adjacent to the nucleotide sequence for binding to Connector B such that binding of both Connector A and Connector B places the connector molecules in a position for ligation of the 5′ end of Connector A to the 3′ end of Connector B.

In some embodiments, the linker domain comprises at least one nucleotide or modified nucleotide that protects the 3′ end from degradation, elongation, ligation, or a combination thereof. In some embodiments, the linker domain comprises at least one, at least two, at least three or more than 3 three terminal uracil ribonucleotides at the 3′-end.

In some embodiments, the linker domain comprises a molecule at the 3′ end which protect the 3′ end and further allows for direct or indirect detection of the glycan binding molecule. Exemplary molecules that can be added to the 3′ end of the glycan binding molecule include, but are not limited to, a fluorescent molecule, biotin, an emission donor molecule, or an emission acceptor molecule. In some embodiments, an exemplary glycan-specific binding molecule comprising a glycan binding domain, spacer, and linker domain comprises SEQ ID NO:13 or SEQ ID NO:33.

### Target Binding Molecule

In some embodiments, the proximity assay of the invention comprises a target binding molecule comprising a) a target binding domain, b) a spacer domain and c) a linker domain specific for binding to the same two connector nucleic acid molecules as the glycan binding domain which, when ligated, serve as a template for rolling circle amplification. Exemplary target nucleic acid molecules that can be detected using the compositions and methods of the invention include glycoRNA molecules, glycoDNA molecules, and glycopeptides.

In some embodiments, the target binding molecule may be a protein, peptide, DNA, RNA, or a chimeric molecule such as an RNA/DNA molecule or a peptide/nucleic acid fusion molecule.

In some embodiments, the target binding domain may be a protein, peptide, DNA, RNA, aptamer, antibody, or a chemical ligand that specifically binds to a target molecule. In some embodiments, the target binding domain binds a single specific target. For example, the target binding domain may be a nucleotide sequence or monoclonal antibody that binds to the target molecule but not to alternative molecules. In some embodiments, the target binding domain binds multiple target sequences. For example, the target binding domain may bind universally to a type of molecules such as an anti-RNA antibody which binds non-specifically to RNA molecules through recognition of the poly A tail.

In some embodiments the target binding domain comprises a nucleotide sequence specific for binding to the nucleic acid portion of a target glycol-nucleic acid molecule. In some embodiments, the target binding molecule is a nucleic acid molecule and the target binding domain comprises 15 to 70 nucleotides at the 5′ end of the target binding molecule.

The spacer in the target binding molecule is generally between the target binding domain and the linker domain. In some embodiments, the spacer domain moves the linker domain away from the target binding domain to enable the linker sequence to come into contact with the connector sequences and subsequent ligation of the connector sequences. In some embodiments, the spacer sequence comprises a flexible sequence 8-15 nucleotides long. In some embodiments, the spacer sequence comprises a poly A sequence 8-15 nucleotides long. In some embodiments, the spacer sequence comprises a poly A sequence 10 nucleotides long. The spacer length can be selected to optimize the proximity ligation distances.

The linker domain of the target binding molecule comprises a) a first nucleotide sequence specific for binding to Connector B in reference to the connector molecules that bind to the linker of the glycan-specific binding molecule described above, and b) a second nucleotide sequence specific for binding to Connector A in reference to the connector molecules that bind to the linker of the glycan-specific binding molecule as described above. In some embodiments, the first nucleotide sequence specific for binding to Connector B comprises 8-20 nucleotides specific for binding to the 5′ end of Connector B. In some embodiments, the second nucleotide sequence specific for binding to Connector A comprises 8-20 nucleotides specific for binding to the 3′ end of Connector A. In some embodiments, the nucleotide sequence for binding to Connector B is directly adjacent to the nucleotide sequence for binding to Connector A such that binding of both Connector B and Connector A places the connector molecules in a position for ligation of the 5′ end of Connector B to the 3′ end of Connector A. In some embodiments, the linker domain of the nucleic acid binding molecule comprises a sequence as set forth in SEQ ID NO: 12, SEQ ID NO: 21, SEQ ID NO:25 or SEQ ID NO:29.

In some embodiments, exemplary nucleic acid binding molecules specific for glycoRNA molecules (comprising a target binding domain, spacer, and linker domain) are set forth in SEQ ID NO:2-10, and SEQ ID NO:34-39.

In some embodiments, the target binding molecule may also include one or more additional moiety. Exemplary additional moieties that can be linked to the target binding molecule of the invention include, but are not limited to a fluorescent molecule, biotin, an emission donor molecule, or an emission acceptor molecule.

### Connector Molecules

In some embodiments, the proximity assay of the invention detects presence of a specific glycosylated molecule by contacting the glycosylated molecule with a combination of a glycan specific binding molecule, a target binding molecule, a first connector DNA molecule (Connector A), and a second connector DNA molecule (Connector B). In some embodiments, Connector A comprises a nucleotide sequence 20-1000 nucleotides in length, wherein 8-30 nucleotides at the 5′ end of Connector A bind to the linker domain of the glycan binding molecule and 8-30 nucleotides at the 3′ end of Connector A bind to the linker domain of the target binding molecule. In some embodiments, Connector B comprises a nucleotide sequence 20-1000 nucleotides in length wherein 8-30 nucleotides at the 3′ end of Connector B bind to the linker domain of the glycan binding molecule and 8-30 nucleotides at the 5′ end of Connector B bind to the linker domain of the nucleic acid binding molecule.

In some embodiments, the connector molecules may also include one or more additional moiety. Exemplary additional moieties that can be linked to the connector molecules of the invention include, but are not limited to a fluorescent molecule, biotin, an emission donor molecule, or an emission acceptor molecule. For example, in some embodiments, Connector A is linked to an emission donor molecule and Connector B is linked to an emission acceptor molecule which provides a detectable signal when Connector A and Connector B are in proximity. In some embodiments, Connector B is linked to an emission donor molecule and Connector A is linked to an emission acceptor molecule which provides a detectable signal when Connector A and Connector B are in proximity.

### Proximity Assay

In some embodiments, the invention provides a method of detecting a target glycosylated molecule in a sample, comprising contacting the sample with a) a glycan binding molecule of the invention, b) a target binding molecule of the invention comprising a target binding domain specific for binding to the target glycosylated molecule, c) a first DNA connector molecule, and d) a second DNA connector molecule. In some embodiments, the method further comprises contacting the sample with a ligation agent for ligation of the first and second DNA connector molecules and detecting the ligated first and second DNA connector molecules.

As used herein, the term “ligation agent” can encompass any number of enzymatic or non-enzymatic reagents. For example, ligase is an enzymatic ligation reagent that, under appropriate conditions, forms phosphodiester bonds between the 3′-OH and the 5′-phosphate of adjacent nucleotides in DNA molecules, RNA molecules, or hybrids. Temperature-sensitive ligases, include, but are not limited to, bacteriophage T4 ligase and E. coli ligase. Thermostable ligases include, but are not limited to, Afu ligase, Taq ligase, Tfl ligase, Tth ligase, Tth HB8 ligase, Thermus species AK16D ligase and Pfu ligase (see for example Published P.C.T. Application WO00/26381, Wu et al., Gene, 76(2):245-254, (1989), Luo et al., Nucleic Acids Research, 24(15): 3071-3078 (1996). The skilled artisan will appreciate that any number of thermostable ligases, including DNA ligases and RNA ligases, can be obtained from thermophilic or hyperthermophilic organisms, for example, certain species of eubacteria and archaea; and that such ligases can be employed in the disclosed methods and kits. Chemical ligation agents include, without limitation, activating, condensing, and reducing agents, such as carbodiimide, cyanogen bromide (BrCN), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of the teachings herein. Detailed protocols for chemical ligation methods and descriptions of appropriate reactive groups can be found in, among other places, Xu et al., Nucleic Acid Res., 27:875-81 (1999); Gryaznov and Letsinger, Nucleic Acid Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucleic Acids Res. 20:3005-09 (1992); Sievers and von Kiedrowski, Nature 369:221-24 (1994); Liu and Taylor, Nucleic Acids Res. 26:3300-04 (1999); Wang and Kool, Nucleic Acids Res. 22:2326-33 (1994); Purmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al., FEBS Letters 232:153-55 (1988); Naylor and Gilham, Biochemistry 5:2722-28 (1966); and U.S. Pat. No. 5,476,930.

In some embodiments, the nucleic acids to be joined comprise the connector DNA molecules and the 5′-phosphorylated and the 3′-hydroxyl ends are ligated when the ends are annealed to the linker domain of the glycan binding molecule or the target binding molecule which functions as a “ligation splint.” In such an embodiment, enzymes including, but not limited to, T4 DNA ligase, Ampligase™. DNA Ligase (Epicentre Technologies, Madison, Wis. USA), Tth DNA ligase, Tfl DNA ligase, or Tsc DNA Ligase (Prokaria Ltd., Reykjavik, Iceland) can be used. However, the invention is not limited to the use of a particular ligase and any suitable ligase can be used.

In some embodiments, the proximity assay includes methods of detecting the ligated DNA connector molecules. There are many suitable methods for the detection of specific nucleic acid sequences and new methods are continually reported. One such category of methods involves specific hybridization reactions of a probe to a junction site formed upon ligation of the two DNA connector molecules. Other methods include generating amplification products and detecting the amplified products using one or multiple probes.

Amplification may refer to any method for increasing the number of copies of a nucleic acid sequence. For example, the amplification may be performed with a polymerase, e.g., in one or more polymerase chain reactions. Amplification may be performed using other suitable methods. These methods often depend on the product catalyzed formation of multiple copies of a nucleic acid or its complement. One of such methods is polymerase chain reaction (PCR), including AFLP (amplified fragment length polymorphism) PCR, allele-specific PCR, Alu PCR, assembly, asymmetric PCR, colony PCR, helicase dependent PCR, hot start PCR, inverse PCR, in situ PCR, intersequence-specific PCR or IS SR PCR, digital PCR, droplet digital PCR, linear-after-the-exponential-PCR or Late PCR, long PCR, nested PCR, real-time PCR, duplex PCR, multiplex PCR, quantitative PCR, or single cell PCR. Other amplification methods may also be used, including ligase chain reaction (LCR), nucleic acid sequence-based amplification (NASBA), linear amplification, isothermal linear amplification, Q-beta-replicase method, 3SR, Transcription Mediated Amplification (TMA), Strand Displacement Amplification (SDA), or Rolling Circle Amplification (RCA).

In some embodiments, ligation of the first and second connector DNA molecules forms a circular product, and the method of detecting the ligated first and second DNA connector molecules includes amplifying the ligated connector molecules using RCA, wherein the 3′ end of the linker domain of the target binding molecule serves as a primer for RCA.

In some embodiments, the ligation of the connector DNA molecules is detected by contacting the ligation product or an amplified product (e.g. RCA product) with a probe specific for binding to the first DNA connector molecule, the second DNA connector molecule or a ligated junction formed from ligation of the first and second DNA connector molecules.

In some embodiments the probe comprises a detectable moiety. For example, useful detectable moieties include 32P, fluorescent dyes, electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, standard superparamagnetic iron oxide (“SSPIO”), SSPIO nanoparticle aggregates, polydisperse superparamagnetic iron oxide (“PSPIO”), PSPIO nanoparticle aggregates, monochrystalline SPIO, monochrystalline SPIO aggregates, monochrystalline iron oxide nanoparticles, monochrystalline iron oxide, other nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable. Any method known in the art for conjugating a probe to a detectable label may be employed to generate a detectable probe for use in the proximity assay of the invention.

In some embodiments, ARPLA can be used for quantitative and/or qualitative detection of a target glycosylated molecule. In some embodiments, quantitative and/or qualitative detection of glycosylated molecule is characterized in that the amplifications are real-time amplifications performed using a labeled probe, for example using a labeled hydrolysis-probe, capable of specifically hybridizing in stringent conditions with a segment of a nucleic acid sequence, or polymorphic nucleic acid sequence. The labeled probe is capable of emitting a detectable signal every time each amplification cycle occurs.

Real-time amplification, such as real-time PCR, can be performed in various different iterations; it can be performed e.g. using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market and are for example commercialized by the Applied Biosystems group (USA). Many fluorescent dyes may be employed, such as FAM dyes (6-carboxy-fluorescein), or any other dye phosphoramidite reagents.

In some embodiments the ligation is detected using an emission donor/acceptor reporter pair. In some embodiments, the glycan specific binding molecule is operably linked to an emission acceptor and the target binding molecule is operably linked to an emission donor or the glycan specific binding molecule is operably linked to an emission donor and the target binding molecule is operably linked to an emission acceptor; and emission of fluorescence only occurs when the emission donor and acceptor are in close proximity. Chemical moieties that emmit fluorescent light operate through a variety of mechanisms, including fluorescence resonance energy transfer (FRET) processes. FRET-based biosensors, composed of fluorophores and sensing domains, have been widely adopted as spectroscopic rulers to monitor a variety of biochemical activities including molecular proximity. FRET donor/acceptor pairs that can be operably linked to the glyco specific binding molecule and target binding molecules of the invention include, but are not limited to, those described in Bajar et al., 2016, Sensors (Basel), 16 (9): 1488.

In some embodiments the ligation is detected using DNA points accumulation for imaging in nanoscale topography (DNA-PAINT) to detect the ligation event which exploits reversible specific binding of labeled oligonucleotides to DNA to provide a detectable signal as described in Jungmann et al., 2010, Nano Lett, 10:4756-4761.

In some embodiments, the ligation is detected using catalytic hairpin assembly (CHA) with a fluorescence signal output as described in Hu et al., 2018, Anal. Chim. Acta, 1042:109-115.

In some embodiments, the ligation is detected using a hybridization chain reaction (HCR) in which multiple labeled oligonucleotides are added which hybridize with the ligated product and then with each other to form a detectable nucleic acid structure as described in Dirks et al., 2004, PNAS, 101:15275-15278.

Although several specific detection methods are described herein, the invention is not limited by the method of detecting the ligation and therefore the assay can be utilized with any methodology for detection of the ligation event.

In some exemplary embodiments, the proximity assay comprises a combination of a glycan binding molecule comprising a glycan binding domain comprising SEQ ID NO: 1 and a linker domain as set forth in SEQ ID NO:12, a target binding molecule comprising a linker domain as set forth in SEQ ID NO:15, a first connector DNA molecule comprising a sequence as set forth in SEQ ID NO:17, a second connector DNA molecule comprising a sequence as set forth in SEQ ID NO:18, and a reporter molecule comprising a sequence as set forth in SEQ ID NO:20.

In some exemplary embodiments, the proximity assay comprises a combination of a glycan binding molecule comprising a sequence as set forth in SEQ ID NO: 33, a target binding molecule comprising a linker domain as set forth in SEQ ID NO: 21, a first connector DNA molecule comprising a sequence as set forth in SEQ ID NO: 22, a second connector DNA molecule comprising a sequence as set forth in SEQ ID NO: 23, and a reporter molecule comprising a sequence as set forth in SEQ ID NO:24.

In some exemplary embodiments, the proximity assay comprises a combination of a glycan binding molecule comprising a sequence as set forth in SEQ ID NO: 33, a target binding molecule comprising a linker domain as set forth in SEQ ID NO: 25, a first connector DNA molecule comprising a sequence as set forth in SEQ ID NO: 26, a second connector DNA molecule comprising a sequence as set forth in SEQ ID NO: 27, and a reporter molecule comprising a sequence as set forth in SEQ ID NO:28.

In some exemplary embodiments, the proximity assay comprises a combination of a glycan binding molecule comprising a sequence as set forth in SEQ ID NO: 33, a target binding molecule comprising a linker domain as set forth in SEQ ID NO: 29, a first connector DNA molecule comprising a sequence as set forth in SEQ ID NO: 30, a second connector DNA molecule comprising a sequence as set forth in SEQ ID NO: 31, and a reporter molecule comprising a sequence as set forth in SEQ ID NO:32.

### Multi-Glycosylated Molecule Analysis

In some embodiments, the invention encompasses methods for detecting multiple target glycosylated molecules in a single assay. In such an embodiment, the assay is designed to detect a panel of at least 2, 3, 4, 5, 6, 7, 8, 9, 10, 11, 12, 13, 14, 15, 20, 25, 30, 35, 40, 45, 50, 60, 70, 80, 90, 100 or more than 100 different glycosylated molecule species in a single reaction. An exemplary panel of glycosylated molecule detection components is one in which each target glycosylated molecule in the panel is detected with a glycan binding molecule and target binding molecule pair that interacts with a specific combination of connector sequences and then is detected with a labeled reporter molecule specific for detecting the ligation event.

For example, in one embodiment, the invention encompasses a method of detecting a combination of:
a) a first glycoRNA wherein the first glycoRNA is detected using a combination of a glycan binding molecule comprising a sequence as set forth in SEQ ID NO: 33, a target binding molecule comprising a linker domain as set forth in SEQ ID NO: 21, a first connector DNA molecule comprising a sequence as set forth in SEQ ID NO: 22, a second connector DNA molecule comprising a sequence as set forth in SEQ ID NO: 23, and a reporter molecule comprising a sequence as set forth in SEQ ID NO:24;
b) a second glycoRNA wherein the second glycoRNA is detected using a combination of a glycan binding molecule comprising a sequence as set forth in SEQ ID NO: 33, a target binding molecule comprising a linker domain as set forth in SEQ ID NO: 25, a first connector DNA molecule comprising a sequence as set forth in SEQ ID NO: 26, a second connector DNA molecule comprising a sequence as set forth in SEQ ID NO: 27, and a reporter molecule comprising a sequence as set forth in SEQ ID NO:28; and
c) a third glycoRNA wherein the third glycoRNA is detected using a combination of a glycan binding molecule comprising a sequence as set forth in SEQ ID NO: 33, a target binding molecule comprising a linker domain as set forth in SEQ ID NO: 29, a first connector DNA molecule comprising a sequence as set forth in SEQ ID NO: 30, a second connector DNA molecule comprising a sequence as set forth in SEQ ID NO: 31, and a reporter molecule comprising a sequence as set forth in SEQ ID NO:32.

In one exemplary embodiment, the multi-glycoRNA panel of the invention detects i) a U1 glycoRNA using the glycan binding molecule as set forth in SEQ ID NO:33, a target binding molecule of SEQ ID NO:34 or SEQ ID NO:35 or a combination thereof, connector DNA molecules as set forth in SEQ ID NO:22 and 23 and a reporter as set forth in SEQ ID NO:24; ii) a U35a glycoRNA using the glycan binding molecule as set forth in SEQ ID NO:33, a target binding molecule of SEQ ID NO:36 or SEQ ID NO:37 or a combination thereof, connector DNA molecules as set forth in SEQ ID NO:26 and 37 and a reporter as set forth in SEQ ID NO:28; and iii) a Y5 glycoRNA using the glycan binding molecule as set forth in SEQ ID NO:33, a target binding molecule of SEQ ID NO:38 or SEQ ID NO:39 or a combination thereof, connector DNA molecules as set forth in SEQ ID NO: 30 and 31 and a reporter as set forth in SEQ ID NO:32.

### Glyco-Omic Profiling

In some embodiments, the assay of the invention can be incorporated into a method for evaluating the glycosylated molecular content of a cell, tissue, or sample in a large scale multi-plex assay (e.g., glycoRNA-omic profile, glycoDNA-omic or glyco-peptide profile). In such an embodiment, tags or barcodes can be added to the linker sequences of the glycan specific binding molecule, the target binding molecule, the connector DNA molecules, or a combination thereof to allow identification of specific target molecules through fluorescent detection using combinations of detectable probes. For example, in some embodiments, the linker sequences or connector DNA sequences incorporate tag or barcode sequences that can be used in multiplexed error-robust FISH (MERFISH) measurements as described in detail in Xia et al., 2019, PNAS, 116:19490-19499 which is incorporated herein by reference in its entirety, for spatial profiling of the glycoRNA, glycoDNA, glycopeptides or a combination thereof.

### Quantitative Algorithm

In some embodiments, the method comprises using a quantitative algorithm to determine if the level of one or more glycosylated molecule in a biological sample is statistically different than the level of the glycosylated molecule in a control sample. The algorithm may be a trained algorithm. In various embodiments, the algorithm is drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; a plurality of algorithms arranged in a committee network; and forward floating search or backward floating search algorithms. Such algorithms may be used in supervised or unsupervised learning modes. In various embodiments, quantitative algorithms according to the disclosure can be used to determine the extent, severity, or stage of disease, to determine the right treatment approach (e.g., disease-specific therapy, surgical intervention), to select the appropriate dose for a medical treatment, to determine whether a patient is likely to respond to a particular medical or surgical treatment, to monitor response to treatment, or to monitor disease progression.

In some embodiments, the methods according to the disclosure include deriving a numerical value, index or score from the quantitative algorithm or mathematical formula. In some embodiments, the derived numerical value can serve as a cut off value for distinguishing between two or more potential outcomes (e.g., high or low risk of disease presence, progression or recurrence or stage of disease.) In some embodiments, a derived numerical value serves as a cutoff value for a level of at least one glycosylated molecule. In some embodiments, a derived numerical value serves as a cutoff value for a level of at least one glycosylated molecule in order determine the extent, severity, or stage of disease. In some embodiments, a derived numerical value serves as a cutoff value for a level of at least one glycosylated molecule in order to determine the right treatment approach (e.g., disease-specific therapy, surgical intervention). In some embodiments, a derived numerical value serves as a cutoff value for an expression level of at least one glycosylated molecule in order to select the appropriate dose for a medical treatment.) In some embodiments, a derived numerical value serves as a cutoff value for a level of at least one glycosylated molecule in order to determine whether a patient is likely to respond to a particular medical or surgical treatment. In some embodiments, a derived numerical value serves as a cutoff value for a level of at least one glycosylated molecule in order to monitor response to treatment. In some embodiments, a derived numerical value serves as a cutoff value for a level of at least one glycosylated molecule in order to monitor disease progression.

### Determining Effectiveness of Therapy or Prognosis

In one aspect, the level of one or more glycosylated molecule in a biological sample of a patient is used to monitor the effectiveness of treatment or the prognosis of disease (e.g., a disease or disorder associated with the inflammatory process or cancer progression). In some embodiments, the level of one or more glycosylated molecule in a test sample obtained from a treated patient can be compared to the level from a reference sample obtained from that patient before initiation of a treatment. Clinical monitoring of treatment typically entails that each patient serves as his or her own baseline control. In some embodiments, test samples are obtained at multiple time points following administration of the treatment. In these embodiments, measurement of level of one or more glycosylated molecule in the test samples provides an indication of the extent and duration of in vivo effect of the treatment.

Measurement of glycosylated molecule levels allow for the course of treatment of a disease to be monitored. The effectiveness of a treatment regimen for a disease can be monitored by detecting one or more glycosylated molecule in an effective amount from samples obtained from a subject over time and comparing the amount of glycosylated molecule detected. For example, a first sample can be obtained before the subject receives treatment and one or more subsequent samples are taken after or during treatment of the subject. Changes in glycosylated molecule levels across the samples may provide an indication as to the effectiveness of the therapy.

In some embodiments, the disclosure provides a method for monitoring the levels of glycosylated molecule in response to treatment. For example, in certain embodiments, the disclosure provides for a method of determining the efficacy of treatment in a subject, by measuring the levels of one or more glycosylated molecule as described herein. In some embodiments, the level of the one or more glycosylated molecule can be measured over time, where the level at one timepoint after the initiation of treatment is compared to the level at another timepoint after the initiation of treatment. In some embodiments, the level of the one or more glycosylated molecule can be measured over time, where the level at one timepoint after the initiation of treatment is compared to the level before initiation of treatment.

In some embodiments, ARPLA can be used to identify therapeutics or drugs that are appropriate for a specific subject. For example, a test sample from the subject can be exposed to a therapeutic agent or a drug, and the level of one or more glycosylated molecule can be determined. Levels of glycosylated molecules can be compared to a sample derived from the subject before and after treatment or exposure to a therapeutic agent or a drug or can be compared to samples derived from one or more subjects who have shown improvements relative to a disease as a result of such treatment or exposure. Thus, in one aspect, the disclosure provides a method of assessing the efficacy of a therapy with respect to a subject comprising taking a first measurement of glycosylated molecule or a glycosylated molecule panel in a first sample from the subject; effecting the therapy with respect to the subject; taking a second measurement of the glycosylated molecule or glycosylated molecule panel in a second sample from the subject and comparing the first and second measurements to assess the efficacy of the therapy.

Accordingly, treatments or therapeutic regimens for use in can be selected based on the amounts of a specific glycosylated molecule or a glycosylated molecule panel in samples obtained from the subjects and compared to a reference value. Two or more treatments or therapeutic regimens can be evaluated in parallel to determine which treatment or therapeutic regimen would be the most efficacious for use in a subject to delay onset, or slow progression of a disease. In various embodiments, a recommendation is made on whether to initiate or continue treatment of a disease.

A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example, prognosis can be expressed as a percent chance that a patient will survive after one year, five years, ten years or the like. Alternatively, prognosis may be expressed as the number of years, on average that a patient can expect to survive as a result of a condition or disease. The prognosis of a patient may be considered as an expression of relativism, with many factors affecting the ultimate outcome. For example, for patients with certain conditions, prognosis can be appropriately expressed as the likelihood that a condition may be treatable or curable, or the likelihood that a disease will go into remission, whereas for patients with more severe conditions, prognosis may be more appropriately expressed as likelihood of survival for a specified period of time. Additionally, a change in a clinical factor from a baseline level may impact a patient's prognosis, and the degree of change in level of the clinical factor may be related to the severity of adverse events. Statistical significance is often determined by comparing two or more populations and determining a confidence interval and/or a p value.

Multiple determinations of glycosylated molecule levels can be made, and a temporal change in glycosylated molecule level can be used to determine a prognosis. For example, comparative measurements are made of the glycosylated molecule level in a patient at multiple time points, and a comparison of the glycosylated molecule level at two or more time points may be indicative of a particular prognosis.

In certain embodiments, the levels of activity of one or more glycosylated molecule level are used as indicators of an unfavorable prognosis. According to the current disclosure, the determination of prognosis can be performed by comparing the measured glycosylated molecule level to levels determined in comparable samples from healthy individuals or to levels corresponding with favorable or unfavorable outcomes. The glycosylated molecule level obtained may depend on a number of factors, including, but not limited to, the laboratory performing the assays, the assay methods used, the type of body fluid sample used and the type of disease a patient is afflicted with. According to the method, values can be collected from a series of patients with a particular disorder to determine appropriate reference ranges of glycosylated molecule levels for that disorder.

In some embodiments the level of glycosylated molecule in a test sample from a patient relates to the prognosis of a patient in a continuous fashion, the determination of prognosis can be performed using statistical analyses to relate the determined glycosylated molecule levels to the prognosis of the patient. A skilled artisan is capable of designing appropriate statistical methods. For example, the methods may employ the chi-squared test, the Kaplan-Meier method, the log-rank test, multivariate logistic regression analysis, Cox's proportional-hazard model and the like in determining the prognosis. Computers and computer software programs may be used in organizing data and performing statistical analyses. The approach by Giles et. al., British Journal of Hemotology, 121:578-585, is exemplary. As in Giles et al., associations between categorical variables (e.g., miRNA levels and clinical characteristics) can be assessed via cross-tabulation and Fisher's exact test. Unadjusted survival probabilities can be estimated using the method of Kaplan and Meier. The Cox proportional hazards regression model also can be used to assess the ability of patient characteristics (such as glycosylated molecule levels) to predict survival, with ‘goodness of fit’ assessed by the Grambsch-Therneau test, Schoenfeld residual plots, martingale residual plots and likelihood ratio statistics (see Grambsch et al, 1995). In some embodiments, this approach can be adapted as a simple computer program that can be used with personal computers or personal digital assistants (PDA). The prediction of patients' survival time in based on their glycosylated molecule levels can be performed via the use of a visual basic for applications (VBA) computer program developed within Microsoft Excel. The core construction and analysis may be based on the Cox proportional hazard models. The VBA application can be developed by obtaining a base hazard rate and parameter estimates. These statistical analyses can be performed using a statistical program such as the SAS proportional hazards regression, PHREG, procedure. Estimates can then be used to obtain probabilities of surviving from one to 24 months given the patient's covariates. The program can make use of estimated probabilities to create a graphical representation of a given patient's predicted survival curve. In certain embodiments, the program also provides 6-month, 1-year and 18-month survival probabilities. A graphical interface can be used to input patient characteristics in a user-friendly manner. In some embodiments of the disclosure, multiple prognostic factors, including glycosylated molecule level, are considered when determining the prognosis of a patient. For example, the prognosis of a cancer patient may be determined based on glycosylated molecule level. For example, in some embodiments, a decrease in glycosylated molecule level is indicative of a worse prognosis for a subject having breast cancer. In some embodiments, a decrease in U1 glycoRNA, U35a glycoRNA, or Y5 glycoRNA, or any combination thereof, is indicative of a worse prognosis for a subject having breast cancer.

In certain embodiments, other prognostic factors may be combined with the glycosylated molecule level or other biomarkers in the algorithm to determine prognosis with greater accuracy. Exemplary additional prognostic factors may include one or more prognostic factors selected from the group consisting of cytogenetics, performance status, age, gender, and contemporary diagnosis.

### Treatments

In one aspect, the disclosure provides a method of diagnosing, treating or preventing a disease or disorder associated with an altered level of a specific glycosylated molecule or a general increase or decrease of total glycosylated molecule, including, but not limited to, diseases or disorders associated with the inflammatory process. In some embodiments, the method comprises administering to the subject an effective amount of a pharmaceutical agent for the treatment of a disease or disorder identified associated with an altered level of a specific glycosylated molecule or a general increase or decrease of total glycosylated molecule, including, but not limited to, diseases or disorders associated with the inflammatory process and cancers.

Exemplary inflammatory diseases and disorder that can be diagnosed, treated or monitored for treatment include, but are not limited to, autoimmune diseases and cancer. Exemplary autoimmune diseases include, but are not limited to, arthritis (e.g., rheumatoid arthritis such as acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, psoriasis vulgaris, inverse psoriasis, erythrodermic psoriasis, seborrheic psoriasis and psoriasis of the nails, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, sclerosis disseminata, and ataxic sclerosis, inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), pyoderma gangrenosum, erythema nodosum, primary sclerosing cholangitis, episcleritis, respiratory distress syndrome, including adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, allergic conditions, allergic reaction, eczema including allergic or atopic eczema, asthma such as asthma bronchiale, bronchial asthma, and auto-immune asthma, conditions involving infiltration of T cells and chronic inflammatory responses, chronic pulmonary inflammatory disease, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis (including large vessel vasculitis (including polymyalgia rheumatica and giant cell (Takayasu's) arteritis), medium vessel vasculitis (including Kawasaki's disease and polyarteritis nodosa), microscopic polyarteritis, CNS vasculitis, necrotizing, cutaneous, or hypersensitivity vasculitis, systemic necrotizing vasculitis, and ANCA-associated vasculitis, such as Churg-Strauss vasculitis or syndrome (CSS)), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, immune complex nephritis, antibody-mediated nephritis, neuromyelitis optica, polyneuropathies, chronic neuropathy such as IgM polyneuropathies or IgM-mediated neuropathy, thrombocytopenia (as developed by myocardial infarction patients, for example), including thrombotic thrombocytopeni purpura (TTP) and autoimmune or immune-mediated thrombocytopenia such as idiopathic thrombocytopenia purpura (ITP) including chronic or acute ITP, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis (e.g., benign monoclonal gammopathy and monoclonal garnmopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine ophthalmopathy, uveoretinitis, chorioretinitis, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia areata, CREST syndrome (calcinosis, Raynaud's phenomenon, esophageal dysmotility, sclerodactyly, and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, fibrosis of any organ or tissue, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, infertility due to antispermatozoan antibodies, non-malignant thymoma, vitiligo, SCID and Epstein-Barr virus-associated diseases, acquired immune deficiency syndrome (AIDS), parasitic diseases such as Leishmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis. Other examples of autoimmune diseases may be disclosed elsewhere herein.

The following are non-limiting examples of cancers that can be diagnosed, treated, or monitored for treatment by the disclosed methods and compositions: acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocvtoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

For example, in some embodiments, a subject having a decrease in glycosylated molecule is diagnosed as having breast cancer. In some embodiments, a subject having a decrease in U1 glycoRNA, U3 glycoRNA, U8 glycoRNA, U35a glycoRNA, or Y5 glycoRNA, or any combination thereof, is diagnosed as having breast cancer. In some embodiments, a subject having a decrease in U1 glycoRNA, U35a glycoRNA, or Y5 glycoRNA, or any combination thereof, is diagnosed as having breast cancer.

Pharmaceutical compositions according to the present disclosure may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present disclosure to be administered can be determined by a physician with consideration of individual differences in age, weight, disease type, extent of disease, and condition of the patient (subject).

Typically, dosages of a compound according to the disclosure which may be administered to an animal range in amount from about 0.01 mg to 20 about 100 g per kilogram of body weight of the animal. While the precise dosage administered will vary depending upon any number of factors, including, but not limited to, the type of animal and type of disease state being treated, the age of the animal and the route of administration. In some embodiments, the dosage of the compound will vary from about 1 mg to about 100 mg per kilogram of body weight of the animal. In some embodiments, the dosage will vary from about 1 μg to about 1 g per kilogram of body weight of the animal. The compound can be administered to an animal as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

### Kits

The present disclosure also pertains to kits useful in the methods of the disclosure. Such kits comprise components useful in any of the methods described herein, including for example, glycan binding molecules, target binding molecules, connecting DNA molecules, reagents for detection of labeled molecules, enzymes, materials and reagents for amplification of a subject's nucleic acids, materials and reagents for ligating DNA molecules, and instructional materials. For example, in some embodiments, the kit comprises components useful for the detection and/or quantification of at least one glycosylated molecule. In some embodiments, the kits can be designed for cellular glycosylated molecule imaging or for quantification of glycosylated molecules in solution.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: GlycoRNA Imaging Via Proximity Ligation Assay Using Aptamer and RNA Probe

A novel imaging method termed ARPLA has been developed to visualize glycoRNA on cell surfaces. ARPLA utilizes a sialic acid aptamer and an in situ hybridization probe for dual recognition of the glycan and RNA portions of the glycoRNA, respectively, followed by a proximity ligation to generate the intact circular DNA, which then serves as a template for RCA to produce ssDNA. The ssDNA can hybridize with numerous copies of fluorophore-labeled reporter DNA to visualize glycoRNA and amplify the signal. Before this report, the state-of-the-art method to detect glycoRNAs is RNA blot using metabolically labeled glycoRNA. Compared with these methods, ARPLA has several advantages: 1) Due to the direct recognition of sialic acid and rapid microscopy imaging, ARPLA can be performed within 4 hours operation time, which is significantly shorter than metabolic labeling (24~72 hours) plus RNA blot (5 hours); 2) ARPLA allows imaging of native, unlabeled glycoRNAs in various type of samples without the need of metabolic labeling used in RNA blot; 3) The fluorescent signals from RCA amplification are strong and discrete (Soderberg, O., et al., 2006, Nature Methods, 3:995-1000), which allows us to study the spatial distribution of glycoRNAs with high sensitivity, whereas other methods cannot provide spatial information; 4) By utilizing two affinity probes, ARPLA can achieve significantly high specificity for glycoRNA instead of the glycan alone, RNA alone or glycan and RNA that are far apart; 5) By replacing the RNA recognition sequence of the ARPLA probe to target different glycoRNAs, ARPLA is quite versatile and customizable to be used to detect and image almost any glycoRNA.

The performance of ARPLA was initially validated in HeLa cells and then the method was applied to investigate the glycoRNA abundance in cancer cell models, and, for the first time, the surface glycoRNA differences were revealed in cancer progression. Specifically, in comparison with non-carcinomatous cell (MCF-10A), malignant breast cancer cell (MCF-7) has a much lower glycoRNA abundance and metastatic breast cancer cell (MDA-MB-231) has the lowest glycoRNA signal. These results indicate an inverse correlation between the glycoRNA and tumor malignant degrees. Furthermore, ARPLA was used to monitor the glycoRNA level during innate immune cells differentiation and activation and uncovered its reduction during immune cell differentiation and increase during LPS induced inflammatory responses. The function of glycoRNAs was studied and it was found that removal of glycoRNA can attenuate the immune cells' attachment to endothelial cells, indicating the potential function of glycoRNA in mediating M-EC interaction during the inflammatory process.

Together, the results reported in this work demonstrate that ARPLA is a novel imaging tool to investigate glycoRNA. Its high sensitivity, selectivity, and its ability to image unlabeled glycoRNAs make it a powerful method to discover and investigate other potential roles of glycoRNAs in many biological processes, including cell communication, host-immune response, RNA protein interactions, and, in the process, uncover their relevance to diseases, such as cancer, autoimmune diseases, and inflammations.

The methods employed are described herein.

### Materials

Sodium chloride, magnesium chloride, calcium chloride, ethylenediaminetetraacetic acid disodium salt dihydrate (EDTA-Na₂), 2-mercaptoethanol, glycerol, NGI-1, Kifunensine, Swainsonine, Chlorofom, dibenzocyclooctyne-PEG4-biotin (DBCO-biotin), and Tween-20 were purchased from Sigma-Aldrich, Inc. (St. Louis, USA). Acid Blue 9 (AB9) was purchased from Fisher Scientific. N-azidoacetylmannosamine-tetraacylated (Ac₄ManNAz) was purchased from Click Chemistry Tools. N-acetylneuraminic acid (Neu5 Ac) was purchased from Cayman Chemical. Bovine serum albumin (BSA), phi29 DNA Polymerase, deoxynucleotide (dNTP), T4 DNA Ligase, adenosine 5′-triphosphate (ATP), Proteinase K, α2-3,6,8,9 Neuraminidase A, PNGase-F, and O-Glycosidase were purchased from New England BioLabs (NEB) Inc. (Ipswich, UK). TRIzol, RNase A, RNase T1, formamide, Hank's Balanced Salt Solution (HBSS), and CellTracker Orange CMRA were purchased from Thermo Fisher Scientific Inc. RNA Clean and Concentrator 5 kit were purchased from Zymo Research. Intercept® (PBS) blocking buffer and IRDye 800CW Streptavidin were purchased from Li-Cor Biosciences. 0.45 μm nitrocellulose membrane was purchased from Cytiva Life Sciences. All the oligonucleotide sequences were purchased from Integrated DNA Technologies, Inc. (USA) and were purified by high performance liquid chromatography (HPLC) or polyacrylamide gel electrophoresis (PAGE) and confirmed by mass spectrometry. All other reagents and solvents were obtained from the domestic suppliers and used as received.

TABLE 1
DNA sequences of used in the experiments (5′ to 3′).
For glycan probes with 3 different sections the Neu5
Ac aptamer sequence is in bold, the space sequence
is underlined, and the DNA linker (Linker G) is
italicized. For RNA binding probes with 3 sections
the RNA in situ hybridization probe (RISH) is in bold,
the spacer sequence is underlined, and the DNA linker
(Linker R) is italicized.

| SEQ | | |
|---|---|---|
| ID NO | Oligonucleotide names | Oligonucleotide sequences |
| 1 | Neu5 Ac aptamer | TAGGGAATTCGTCGACGGATCCCGTGG |
| | | CGTCTGCAACGGAAAAGAATTTATCTT |
| | | GTCCTGCAGGTCGACGCATGCGCCG |
| 2 | RNA binding probe for Ul | CTGGGAAAACCACCTTCGTGATCATG |
| | glycoRNA - 1 | GTATCTCCCCTGCCAGGTAAGTATAA |
| | | AAAAAAAATATGACAGAACTAGACACTC |
| | | TT |
| 3 | RNA binding probe for Ul | CGAACGCAGTCCCCCACTACCACAAA |
| | glycoRNA - 2 | TTATGCAGTCGAGTTTCCCACATTAA |
| | | AAAAAAAATATGACAGAACTAGACACTC |
| | | TT |
| 4 | RNA binding probe for U3 | TCTTCCTCGTGGTTTTCGGTGCTCTA |
| | glycoRNA - 1 | CACGTTAAAAAAAAAATATGACAGAAC |
| | | TAGACACTCTT |
| 5 | RNA binding probe for U3 | CTCCCCAATACGGAGAGAAGAAAAA |
| | glycoRNA - 2 | AAAAATATGACAGAACTAGACACTCTT |
| 6 | RNA binding probe for U8 | GTTCTAATCTGCCCTCCGGAGGAGG |
| | glycoRNA | AACAGGTAAGGATTATCCCACCAAAA |
| | | AAAAAATATGACAGAACTAGACACTCTT |
| 7 | RNA binding prob for U35a | AGACCATCGTGAGATAAGAAAAAAA |
| | glycoRNA - 1 | AAATATGACAGAACTAGACACTCTT |
| 8 | RNA binding probe for U35a | CTCCTGGCATCAGCTAAGAAAAAAAA |
| | glycoRNA - 2 | AATATGACAGAACTAGACACTCTT |
| 9 | RNA binding probe for Y5 | GGGAGACAATGTTAAATCAAAAAAA |
| | glycoRNA - 1 | AAATATGACAGAACTAGACACTCTT |
| 10 | RNA binding probe for Y5 | AAAACAGCAAGCTAGTCAAAAAAAAA |
| | glycoRNA - 2 | ATATGACAGAACTAGACACTCTT |
| 11 | RNA binding probe w/o | AAAAAAAAAATATGACAGAACTAGACAC |
| | RISH-R | TCTT |
| 12 | RNA binding probe linker | TATGACAGAACTAGACACTCTT |
| | domain | |
| 13 | Glycan probe | TAGGGAATTCGTCGACGGATCCCGT |
| | | GGCGTCTGCAACGGAAAAGAATTTA |
| | | TCTTGTCCTGCAGGTCGACGCATGC |
| | | GCCGAAAAAAAAAAGACGCTAATAGTT |
| | | AAGACGCTTUUU (3 terminal uracil |
| | | ribonucleotides) |
| 14 | Glycan probe | AAAAAAAAAAGACGCTAATAGTTAAGAC |
| | w/o Aptamer-G | GCTTUUU |
| | | (3 terminal uracil ribonucleotides) |
| 15 | Glycan probe | GACGCTAATAGTTAAGACGCTT |
| | linker domain | |
| 16 | Glycan probe | CGGCGCATGCGTCGACCTGCAGGAC |
| | using DNA with | AAGATAAATTCTTTTCCGTTGCAGAC |
| | scrambled sequence | GCCACGGGATCCGTCGACGAATTCC |
| | | CTAAAAAAAAAAAGACGCTAATAGTTAA |
| | | GACGCTTUUU (3 terminal uracil |
| | | ribonucleotides) |
| 17 | Connector 1 | Phosphate- |
| | | CTATTAGCGTCCAGTGAATGCGAGTCC |
| | | GTCTAAGAGAGTAGTACAGCAGCCGTC |
| | | AAGAGTGTCTA |
| 18 | Connector 2 | Phosphate- |
| | | GTTCTGTCATATTTAAGCGTCTTAA |
| 19 | poly T oligonucleotides | TTTTTTTTTTTTTTTTTTTTTTT |
| 20 | Reporter | Alexa 647-CAGTGAATGCGAGTCCGTCT |

TABLE 2
DNA Sequences used for multiple glycoRNAs imaging (5′ to 3′)

| SEQ ID | Oligonucleotide | |
|---|---|---|
| NO | names | Oligonucleotide sequences |
| 21 | Mul-A ligation | CTGACATACTGTCCTAGCGATT |
| | domain | |
| 22 | Mul-A-Connect- | CAGTATGTCAGCCCAGAG |
| | S | |
| 23 | Mul-A-Connect- | CGTCCAAATGATTTTCAGTGAATGCGAGTCCGT |
| | L | CTAAGAGAGTAGTACAGCAGCCGTCAATCGCTA |
| | | GGA |
| 24 | Mul-A reporter | /5Cy5/AATCGCTAGGACAGTATGTCAG |
| 25 | Mul-B ligation | CCGAATAGGTGCTTTGAAGCAC |
| | domain | |
| 26 | Mul-B-Connect- | CCGAATAGGTGCTTTGAAGCAC |
| | S | |
| 27 | Mul-B-Connect- | CGTCCAAATGATTTTCAGTGAATGCGAGTCCGT |
| | L | CTAAGAGAGTAGTACAGCAGCCGTCGTGCTTCA |
| | | AAG |
| 28 | Mul-B reporter | /36-TAMTSp/GTGCTTCAAAGCACCTATTCGG |
| 29 | Mul-C ligation | GCCCTTGTTAAGACCATTTGTG |
| | domain | |
| 30 | Mul-C-Connect- | TTAACAAGGGCCCCAGAG |
| | S | |
| 31 | Mul-C-Connect- | CGTCCAAATGATTTTCAGTGAATGCGAGTCCGT |
| | L | CTAAGAGAGTAGTACAGCAGCCGTCCACAAATG |
| | | GTC |
| 32 | Mul-C reporter | /56-FAM/CACAAATGGTCTTAACAAGGGC |
| 33 | Mul-glycan | TAGGGAATTCGTCGACGGATCCCGTGGCGTCTG |
| | | CAACGGAAAAGAATTTATCTTGTCCTGCAGGTC |
| | | GACGCATGCGCCGAAAAAAAAAATCATTTGGAC |
| | | GCTCTGGGUUU |
| | | (3 terminal uracil ribonucleotides) |
| 34 | Mul-U1-FISH-1 | CTGGGAAAACCACCTTCGTGATCATGGTATCTC |
| | | CCCTGCCAGGTAAGTATAAAAAAAAAACTGACA |
| | | TACTGTCCTAGCGATT |
| 35 | Mul-U1-FISH-2 | CGAACGCAGTCCCCCACTACCACAAATTATGCA |
| | | GTCGAGTTTCCCACATTAAAAAAAAAACTGACA |
| | | TACTGTCCTAGCGATT |
| 36 | Mul-U35a-FISH- | AGACCATCGTGAGATAAGAAAAAAAAAACCGA |
| | 1 | ATAGGTGCTTTGAAGCAC |
| 37 | Mul-U35a-FISH- | CTCCTGGCATCAGCTAAGAAAAAAAAAACCGAA |
| | 2 | TAGGTGCTTTGAAGCAC |
| 38 | Mul-Y5-FISH-1 | GGGAGACAATGTTAAATCAAAAAAAAAAGCCC |
| | | TTGTTAAGACCATTTGTG |
| 39 | Mul-Y5-FISH-2 | AAAACAGCAAGCTAGTCAAAAAAAAAAGCCCT |
| | | TGTTAAGACCATTTGTG |

### Isothermal Titration Calorimetry (ITC)

To study the binding affinity between Neu5 Ac and its aptamer, ITC was performed using a VP-ITC microcalorimeter instrument (MicroCal). Neu5Ac and aptamer were dissolved in 1×aptamer binding buffer (50 mM Tris-HCl, 5 mM KCl, 100 mM NaCl and 1 mM MgCl₂, pH 7.4), respectively. Before ITC analysis, the pH of Neu5Ac and aptamer solutions were carefully titrated to pH 7.4, which was very important for the successful ITC analysis. The solution of Neu5Ac and its aptamer were degassed for 10 min prior to applying them for ITC. 20 μM Neu5 Ac aptamer were loaded in the cell. 1 mM Neu5Ac in the same buffer was loaded into the syringe. After the first injection of 2 μL, the syringe injected 10 μL of the Neu5Ac into the cell each time. Through measuring the heat changes and fitting the titration curves to a one-site binding model, thermodynamic data, including the binding constant (K_{d}), enthalpy change (ΔH), entropy change (ΔS), free energy change (ΔG), and binding stoichiometry (N) were obtained. The molar ratio was calculated from the ITC data based on the Neu5 Ac/aptamer concentrations.

### Cell Culture

MDA-MB-231 was a gift from Dr. Aaron Baker (the University of Texas at Austin. SH-SY5Y cell line was obtained from Dr. Liviu Mirica (the University of Illinois at Urbana-Champaign). The THP-1 cell line was obtained from Dr. Jefferson Chan (the University of Illinois at Urbana-Champaign). All other cell lines were purchased from American Type Culture Collection (ATCC, Manassas, VA), Cancer Center at the University of Illinois at Urbana-Champaign, or PromoCell and were cultured at 37 °C in a humidified incubator with 5% CO₂.

HeLa, HEK293T, and PANC-1 cell lines were cultured in Dulbecco's modification of Eagle's medium (DMEM) supplemented with 10% fetal bovine serum (FBS), 100 U/mL penicillin, and 100 U/mL streptomycin. The human neuroblastoma cell line SH-SY5Y was cultured in a 1:1 mixture of Eagle's Minimum Essential Medium (EMEM) and Ham's F12 Medium, supplemented with 10% FBS, 100 U/ml penicillin, and streptomycin.

Non-tumorigenic breast cell line MCF-10A (human mammary gland epithelia) were cultured in 1:1 DMEM/F12 supplemented with 5% horse serum, 20 ng/ml EGF, 0.5 μg/ml hydrocortisone, 10 μg/ml insulin and 100 U/mL penicillin/streptomycin. Breast cancer cell lines (malignant MCF-7 cells and metastatic MDA-MB-231 cells) were cultured in DMEM with 100 U/mL of penicillin, 100 U/mL of streptomycin, 1×non-essential amino acids (NEAA) and 10% FBS.

The human monocyte cell line THP-1 was cultured in RPMI-1640 medium supplemented with 2.5 mM glutamine, 1×MEM NEAA and 10% heat-inactivated FBS. To differentiate THP-1 to macrophage-like cells, the cells were treated with 250 nM phorbol 12-myristate 13-acetate (PMA) in the 10% FBS culture medium for 24 h and then treated with the medium RPMI-1640 containing 5% FBS for another 2 days (Baxter, E. W., et al., 2020, Journal of Immunological Methods, 478:112721). To activate the resting macrophage, the macrophages were incubated with 5% FBS RPMI-1640 medium containing 12.5 μg/ml lipopolysaccharide (LPS) overnight.

The human promyelocytic leukemia cell line HL-60 was cultured in growth media consisting of Iscove's Modified Dulbecco's medium (Corning), supplemented with 20% FBS (Fisher), 1×GlutaMAX, and 1×MEM NEAA. The cells were differentiated into neutrophil-like cells (dHL-60) with a 5-day treatment of 1.3% dimethyl sulfoxide (DMSO) (Sigma, St. Louis, MO) in growth media (Tarella, C., et al., 1982, Cancer Research, 42:445-449; Millius, A., et al., 2010, Humana Press, 147-158).

Human umbilical vein endothelial cells (HUVEC) were purchased from PromoCell and cultured in Endothelial Cell Media 2 (PromoCell). For the subculture of HUVEC, 2 μg/cm² of human plasma fibronectin (EMD Millipore) was coated to culture flasks for 2 h in the incubator.

### Blotting Analysis of Ac₄ManNAz-Labeled RNA

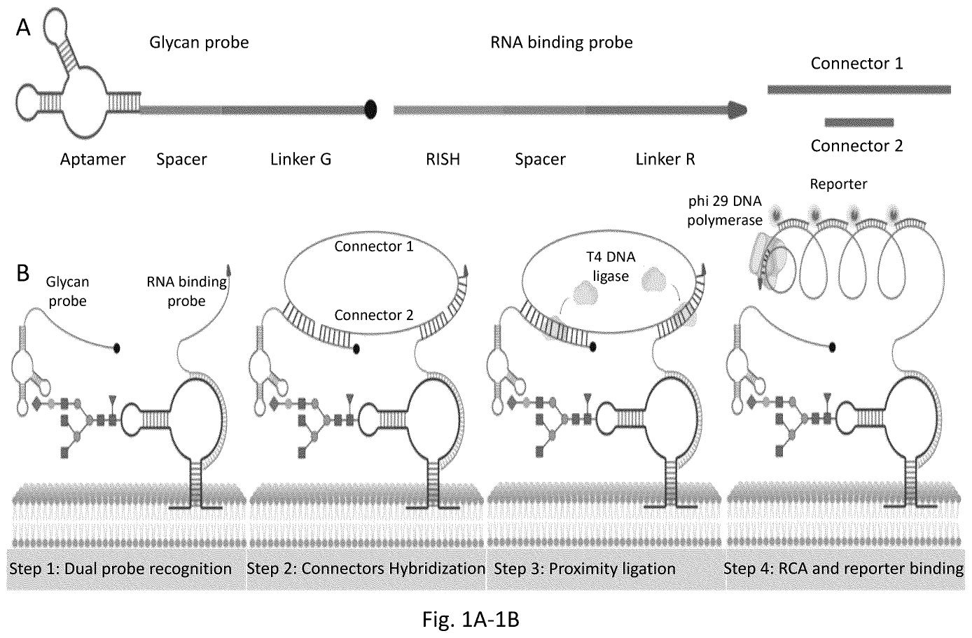

GlycoRNAs of HeLa cells and other cell lines were verified by metabolic labeling with Ac₄ManNAz, followed by RNA blot assay to confirm the presence of glycoRNA as described in a previous report (Flynn, R. A., 2021, Cell, 184:3109-3124). Briefly, after metabolic labeling, the total RNA was extracted, stripped of protein contamination via proteinase K digestion, and finally cleaned up with silica columns. The purified RNA was then labeled with DBCO-biotin via biorthogonal click chemistry and subsequently analyzed by denaturing gel electrophoresis and RNA blot. As shown in Extended Data FIG. 1c, the total RNA extracted from metabolically labeled HeLa cells possess significant biotinylated species. In contrast, for HeLa cells without metabolic labeling treatment, its total RNA was also extracted and incubated with Ac₄ManNAz in test tube, and no detectable signal was observed (Extended Data FIG. 1c). Therefore, Ac₄ManNAz was metabolically incorporated into glycan moiety of glycoRNA, demonstrating the presence of glycoRNA in HeLa cells.

### Metabolic Labeling

For the RNA blot experiments and metabolic labeling, cells were treated with 100 μM Ac₄ManNAz in cell culture media for 72 hours.

### RNA Extraction and Purification

The RNA extraction and purification were performed according to a previously reported method (Flynn, R. A., et al., 2021, Cell, 184:3109-3124). First, the cells were treated with 2 mL TRIzol reagent and incubated at 37 °C to denature non-covalent interactions. Then, phase separation was performed by adding 0.4 mL of chloroform, vortex, incubation for 5 min, and spinning at 12,000×g at 4 °C for 15 min. The RNA in the aqueous phase was carefully transferred to a fresh tube and purified by a Zymo RNA Clean and Concentrator 5 kit from Zymo Research. Thereafter, to avoid protein contamination, the obtained RNA was subjected to protein digestion by adding 1 μg of Proteinase K to 25 μg of purified RNA in 30 mM Tris HCl (pH 8.0) and incubated at 37 °C for 45 min. Finally, to remove the proteinase K, the RNA was purified again with RNA Clean and Concentrator 5 kit and stored at −80 °C for future application.

### Copper-Free Click Conjugation to Ac₄ManNAz-Labeled RNA

50 μL Ac₄ManNAz-labeled RNA extracted from the cells were mixed with 47.5 μL RNA denature buffer (95% formamide, 18 mM EDTA, and 0.025% SDS) and 2.5 μL of 20 mM DBCO-biotin. The conjugation was performed at 55 °C for 10 min in Thermomixer. Therefore, biotin-labeled RNA was purified by Zymo RNA Clean and Concentrator 5 kit.

### RNA Gel Electrophoresis, Blotting, and Imaging

The blotting analysis of biotin-labeled RNA was performed according to a previously reported method (Flynn, R. A., et al., 2021, Cell, 184:3109-3124). The biotin-labeled RNA was lyophilized dry and subsequently resuspended in 15 μL of RNA denature buffer, and then incubated at 55 °C for 10 min and cooled down on ice. Thereafter, 10 μg RNA samples were loaded into 1% agarose-formaldehyde denaturing gel, electrophoresed at 110 V for 1 hour at 4 °C, and finally visualized after SYBR Safe staining. Then, the RNA sample was transferred to 0.45 μm nitrocellulose (NC) membrane at 20 V for 30 min at 4 °C. Afterwards, the RNA sample was crosslinked to the NC membrane with UV crosslinker (Fisher Scientific) for 5 min. Then, the NC membrane was blocked with Intercept® (PBS) blocking buffer at 25 °C for 45 min. Thereafter, the NC membrane was stained with IRDye 800CW Streptavidin in Intercept® (PBS) blocking buffer (1:10,000 dilution) at 25 °C for 30 min. Then, the NC membrane was washed with 1×PBS containing 0.1% Tween-20 for 3 times, and 1×PBS once. Finally, the NC membrane was scanned with an Odyssey Li-Cor CLx scanner (Li-Cor Biosciences) with the software set to auto-detect the signal intensity for the 800 channels.

In Situ Imaging of glycoRNA with ARPLA

### Surface-RNA In Situ Hybridization

HeLa, HEK293T, SH-SY5Y, PANC-1, MCF-10A, MCF-7, and MDA-MB-231 cells were seeded into glass-bottom 35 mm imaging dishes (MetTek) at the density of 2×10⁵ cells/well. HL-60, dHL-60, THP-1, resting macrophage (M0), and activated M0 macrophage were seeded in the same day, at the density of 1×10⁶ cells/well, rest and let the cells attach in serum-free RPMI-1640 for 30 min prior treatment.

All the cell samples were washed three times with PBS without Mg²⁺ and Ca²⁺ before each step. First, the cells were fixed with 4% paraformaldehyde (PFA) at 37 °C for 15 min. To block the non-specific interaction, the cells were blocked with 100 nM poly T oligonucleotides and 0.25 μg/μL BSA in 1×hybridization buffer (50 mM Tris-HCl buffer, 10 mM MgCl₂, pH 7.4) at 37 °C for 30 min. Note that no permeation was performed. Thereafter, the cells were incubated with 1.5 μM RNA binding probe, 0.25 μg/μL BSA, 250 mM NaCl, 1×hybridization buffer at 37 °C for 30 min. After surface-RNA in situ hybridization, the cells were subsequently washed 3 times in FISH washing buffer (2×SSC, 10% formamide) for 10 min each time, followed by 3 times wash in PBS to remove residual formamide.

Aptamer Recognize Neu5Ac in glycoRNA and Proximity Assisted In Situ Ligation

First, the cells were incubated with 100 μL of aptamer and connector solution. The solution contains 100 nM of Neu5 Ac aptamer, 0.25 μg/μL BSA, 100 nM poly T oligonucleotides, 125 nM connector 1, 125 nM connector 2, 1×aptamer binding buffer (50 mM Tris-HCl, 5 mM KCl, 100 mM NaCl and 1 mM MgCl₂, pH 7.4). The cells were incubated with this solution at 37 °C for 30 min, ensuring aptamer binding to Neu5 Ac on glycoRNA, and then assisting the hybridization of connector 1, 2.

Thereafter, the ligation mixture was added to the aptamer and connector solution in the cell dish and mixed well by pipetting. The final solution contained 1 U/μL T4 DNA ligase, 1 mM ATP, 1×T4 DNA ligase reaction buffer provided by NEB.

### In Situ RCA Reaction

The in-situ RCA reaction was performed with RCA working solution at 37 °C for 90 min, which contains 2.5 U/μL phi29 DNA polymerase, 0.25 mM dNTP, 0.2 μg/μL BSA, 5% glycerol, 1×phi29 DNA polymerase reaction buffer provided by NEB. Finally, the single stranded RCA products were detected by in situ hybridization with 100 nM reporter in 2×SSC buffer, 0.25 μg/μL BSA, 100 nM poly T oligonucleotides at 37 °C for 30 min. From this step onward, the slide was kept in the dark. The slides were mounted with mounting media, and pictures were taken with a confocal microscope.

### Microscopy and Image Analysis

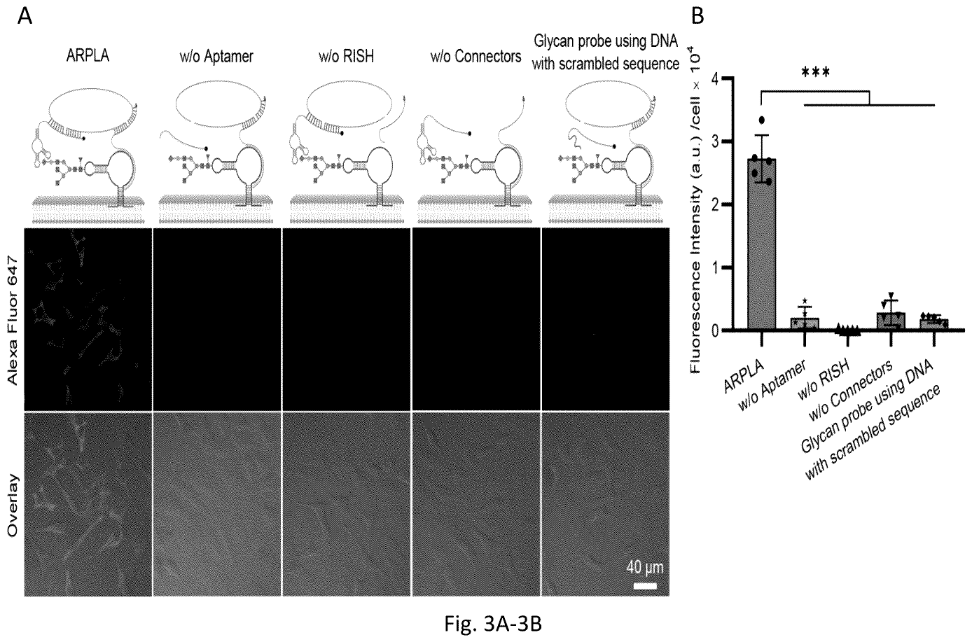

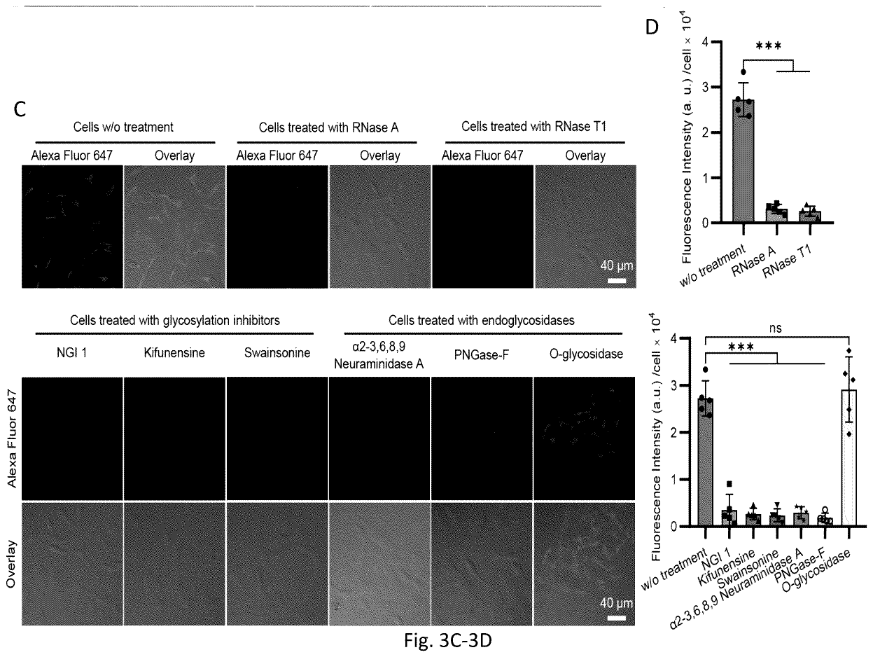

The images were taken with Nikon W1 spinning disk microscope (Nikon). To accomplish the imaging, 60×water-immersion objective was applied, and the fluorophore was excited with a 640 nm laser and Cy5 filter (Em 672-712 nm). The images were taken with monochromatic Andor EMCCD cameras and were processed using ImageJ. Amplicons with a bright fluorescent signal were distinguished from the background by adjusting the threshold. The average fluorescence intensity of all cells in each frame was calculated by ImageJ. And more than 5 frames of each imaging group were processed for further statistical analysis. >3 biological replicates were performed and validated, showing similar trends. In FIG. 3b, the relative fluorescence intensity was calculated by comparing the signal from each group with the group showing the highest fluorescence signal.

### Validation of the Specificity of ARPLA

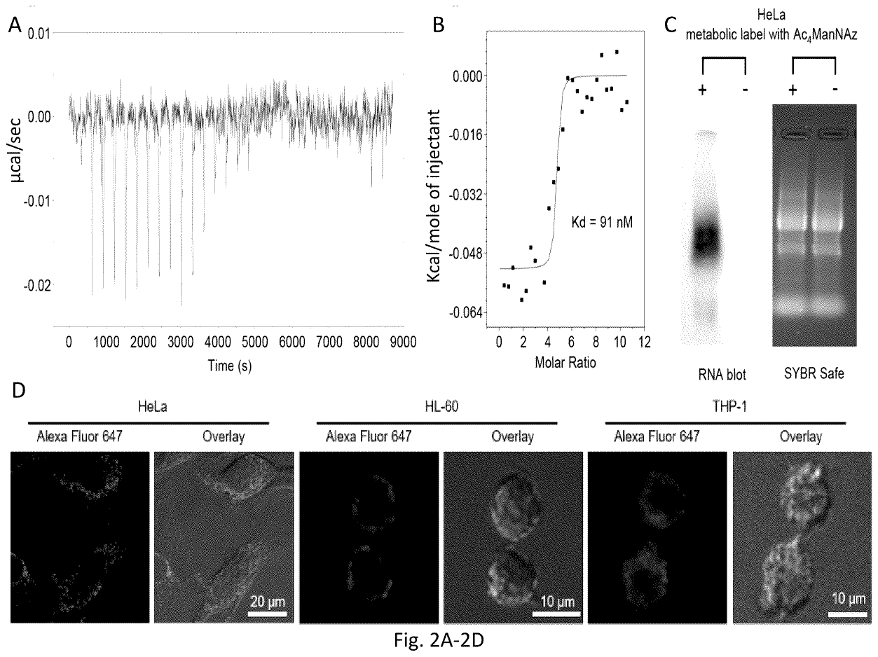

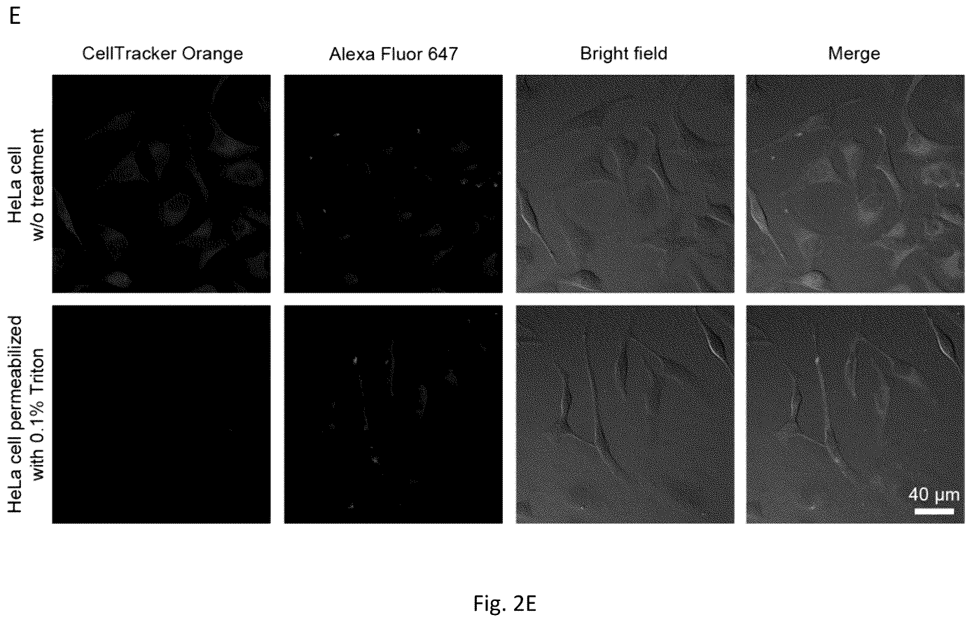

To validate the specificity of ARPLA, several treatments were applied to modulate the levels of RNA or glycan moiety of the glycoRNA to test whether ARPLA can specifically detect glycoRNA to differentiate it from other cell surface RNA or glycan. To digest the RNA moiety of glycoRNA and then validate the sensor performance, the cells were incubated with 0.02 μg/μL RNase A or 1 U/μL RNase T1 in 100 μL HBSS at 37 °C for 20 min and then analyzed by ARPLA. As shown in FIG. 2c,d, after RNase A or RNase T1 treatment, the fluorescence signals were reduced to nearly 12% or 10% of that of the cells without RNase treatment.

Moreover, to verify the ARPLA's response to the glycan moiety of glycoRNA, pharmacological and enzymatic methods were utilized, respectively, to remove the glycan moiety. In the pharmacological approach, the cells were pretreated either 1) 8 μM N-linked Glycosylation Inhibitor 1 (NGI-1), which is a specific small molecule inhibitor of Oligosaccharyltransferase related to glycoRNA generation⁵; 2) 2 μM Kifunensine, which can inhibit N-glycan processing⁶; or 3) 40 μM Swainsonine, which can perturb N-glycan processing (Lopez-Sambrooks, C., et al., 2016, Nature Chemical Biology, 12:1023-1030; Elbein, A. D., et al., 1990, Journal of Biological Chemistry, 265:15599-15605; Tulsiani, D. R., 1982, Journal of Biological Chemistry, 257:7936-7939). The stock solution of glycan-biosynthesis inhibitors, NGI-1, Kifunensine, and Swainsonine, were all made in DMSO at the concentrations of 10 mM and stored at −80 °C. Then 8 μM NGI-1, 2 μM Kifunensine, or 40 μM Swainsonine were incubated with HeLa cells, respectively, for 24 h before the glycoRNA imaging experiment. In the enzymatic method, to digest the glycan on the living cell surface, the HeLa cells were incubated with PNGase-F, α2-3,6,8,9 Neuraminidase A or O-glycosidase at the concentration of 10 U/100 μL in HBSS at 37 °C for 30 min, respectively. Then the cells were analyzed by ARPLA.

### Lipid Raft Staining and Immunofluorescent Imaging

To label lipid rafts, two lipid raft-specific dyes were utilized, Vybrant™ Alexa Fluor™ 555 Lipid Raft Labeling Kit (Invitrogen) and BODIPY™ FL C₅-Ganglioside G_{M1} (Invitrogen). Product manuals were followed to label lipid rafts before seeding the cells to imaging plates. In brief, for the Vybrant™ Alexa Fluor™ 555 Lipid Raft Labeling Kit, cells were pelleted and washed with pre-cold RPMI supplemented with 10% FBS three times and resuspended the cells in 4×10⁶ cells/mL. 2 μL of cholera toxin subunit B stock solution (1 mg/mL) was added to 2 mL cell solution. The cells were incubated on ice for 15 min and then washed twice with pre-cold PBS. Thereafter, cells were resuspended in serum-free RPMI and seeded into imaging dishes. After cells adhesion, the cells were fixed with 4% PFA without cell membrane permeabilization and then stained by with ARPLA. For BODIPY™ FL C₅-Ganglioside G_{M1} staining, cell pellets were washed with pre-cold HBSS, resuspend in 2×10⁶ cells/100 μl of 5 uM Ganglioside GM1 working solution. The cells were incubated on ice for 30 mins, and then washed with pre-cold HBSS for 3 times and resuspend in serum-free RPMI for seeding. After cell adhesion, the cells were fixed and analyzed with ARPLA.

The following antibodies were used to stain SNARE proteins, TSNARE1 Polyclonal Antibody (Invitrogen), VTI1B Polyclonal Antibody (Invitrogen), and Donkey anti-Rabbit IgG (H+L) Highly Cross-Adsorbed Secondary Antibody (Invitrogen). The HL-60 cells were washed twice with serum-free RPMI, and then resuspend in serum-free RPMI at the density of 1×10⁶ cells/mL and seeded into 35 mm glass-bottom confocal imaging dishes. The cells were fixed with 4% PFA solution at room temperature for 15 min, and then incubate with 0.2% Triton X-100 solution for 5 min at RT. After washing with PBS, the cells were stained ARPLA with minor revision. Prior to add Reporter strand, 1:250 diluted 1^{st} antibody solutions (prepared in 1% BSA) were applied to the cells and incubate at 4 °C overnight. After washing cells with PBS for 5 times, we applied 1:500 diluted 2^{nd} antibody solution (prepared in 1% BSA) to cells and stain at RT for 1 h. We washed cells for 3 times and then added ARPLA Reporter strand and incubate at 37 °C for 30 min, washed twice with PBS and mounted the cells in mounting media.

Fluorescent images were taken with W1 Nikon Spinning Disk confocal microscopy. Then the images were processed with Nikon NIS Element viewer. The colocalization assay was performed with Coloc2 plug-in of FIJI (ImageJ). And the plot profiles were also analyzed by using FIJI.

### Transmission-Through-Dye Image of HeLa Cell

The membrane permeable dye (CellTracker Orange CMRA) and the membrane impermeant quencher acid blue 9 (AB9) were applied to the same cells. Since AB9 cannot enter the cell with an intact membrane and thus cannot quench the dye, the cells with an intact membrane will have bright fluorescence signals from CellTracker Orange CMRA. However, a leaky or damaged membrane after permeabilization treatment allows for the quencher to enter the cell, resulting in reduced or diminished fluorescence signal of the cell.

HeLa cells were seeded into glass-bottom 35 mm imaging dishes one day prior to treatment, at the density of 0.6×10⁶ cells/well. Then the cells were stained with CellTracker Orange CMRA at the concentration of 5 μM for 30 min. Afterward, HeLa cells were treated with all the procedures of ARPLA. Then, HeLa cells were stained with AB9 at the concentration of 5 mg/mL for 30 min, then imaged by a confocal microscope. The negative control cells with broken cell membranes were prepared by permeabilization with 0.1% Triton.

### Cell Attachment Assay

THP-1, resting M0, LPS activated M0 macrophage, HL-60, and dHL-60 cells were stained with CellTracker Orange CMRA (5 μM) for 30 min at 37 °C for live-cell fluorescent tracking. Afterwards, cells were washed and resuspended in HBSS at 1×10⁶ cells/mL. To digest the RNA on the living cell surface, the cells were treated with 0.02 μg/μL RNase A and 1 U/μL RNase T1 in PBS at 37 °C for 30 min. After incubation, the cells were washed thoroughly in PBS three times and were seeded onto a confluent layer of HUVECs in 96-well plates prepared 48-72 h before the experiment. After cell attachment, unattached cells were removed with HBSS washes. Attachment of the fluorescently labeled cells was quantified using the ZEISS Observer 7 fluorescence microscopy. The number of attached cells was calculated using ImageJ, which counts cells with a bright fluorescent signal distinguished from the background by adjusting the threshold. The absolute cell number was quantified using the particle analysis with parameters 0.5-15 pixel (Tarella, C., et al., 1982, Cancer Research, 42:445-449).

### Data Analysis

All experiments were performed with at least 3 biological replicates. For each individual biological replicate, 3 technical repeats were performed in cell imaging experiments, and 6 technical repeats were performed in cell attachment assay. Each test was displayed as the mean±standard deviation (SD). For comparison of two independent groups, two-tail unpaired Student's t-test were performed. The statistical significance is determined by the t test as (ns) not significant, (*) P<0.05, (**) P<0.01, and (***) P<0.001.

The Experimental results are now described.

To address the limitations in the current glycoRNA detection methods a novel method has been developed to directly visualize glycoRNA on the cell surface with high sensitivity and selectivity, utilizing ARPLA that contains a sialic acid aptamer for glycan-binding and a DNA-based in situ hybridization probe to target the RNA of the glycoRNA. Aptamers are single-stranded nucleic acids that act as affinity reagents for different targets, and they have been widely applied for bioimaging, diagnostics, sensors, and therapeutics. The key to ARPLA is the in situ proximity ligation assay, which depends on two binding probes that are required to be close to each other as the prerequisite to generate a signal (FIG. 1) (Soderberg, O., et al., 2006, Nature Methods, 3:995-1000; Tavoosidana, G., et al., 2011, Proceedings of the National Academy of Sciences USA, 108:8809-8814; Jalili, R., et al., 2018, Proceedings of the National Academy of Sciences USA, 115: E925-E933; Dore, K., et al., 2020, Proceedings of the National Academy of Sciences USA, 117:3214-3219). Benefitting from the dual-recognition in proximity to prevent false positive signals from recognizing either target individually or both targets that are far apart, proximity ligation assays have been widely applied to monitor the modifications and distribution of biomacromolecules that have either more than one recognizable moiety or can interact with another partner (Soderberg, O., et al., 2006, Nature Methods, 3:995-1000; Weibrecht, I., et al., 2013, Nature Protocols, 8:355-372; Gao, X., et al., 2014, Nature Protocols, 9:2607-2623; tom Dieck, S., et al., 2015, Nature Methods, 12:411-414; Robinson, P. V. et al., 2016, Journal of the American Chemical Society, 138:10722-10725; Frei, A. P., et al., 2016, Nature Methods, 13:269-275; Duckworth, A. D., 2019, Nature Protocols, 14:901-920; Ohata, J., et al., 2020, ACS Central Science, 6:32-40; Ren, X., et al., 2021, Angewandte Chemie International Edition English, 60:22646-22651). In ARPLA, the dual recognition of the glycan and RNA moiety in glycoRNA is achieved by combining an aptamer for glycan-binding and a DNA probe for RNA in situ hybridization (Yue, H., et al., 2021, Sensors & Actuators B: Chemical, 344:130270). The dual recognition in ARPLA ensures high selectivity for only glycoRNA and provides specific RNA sequence information. The high sensitivity of the detection is achieved when the dual recognition probe triggers in situ ligation and then rolling circle amplification (RCA) to generate signal outputs by the binding of fluorophore-labeled DNA probes (Soderberg, O., et al., 2006, Nature Methods, 3:995-1000). The state-of-the-art method to detect glycoRNAs is RNA blot using metabolically labeled glycoRNA. Compared with these methods, ARPLA has several advantages: 1) The fluorescent signals from RCA amplification are strong and discrete, which allows us to study the spatial distribution of glycoRNAs with high sensitivity, whereas other methods cannot provide spatial information; 2) Due to the direct recognition of sialic acid and rapid microscopy imaging, ARPLA can be performed within 4 hours operation time, which is significantly shorter than metabolic labeling (24~72 hours) plus RNA blot (5 hours); 3) ARPLA allows imaging of native, unlabeled glycoRNAs in various sample types without the need for metabolic labeling used in RNA blot; 4) By utilizing two affinity probes, ARPLA can achieve significantly high specificity for glycoRNA instead of the glycan alone, RNA alone or glycan and RNA that are far apart; 5) By replacing the RNA recognition sequence of the ARPLA probe to target different glycoRNAs, ARPLA is quite versatile and customizable to be used to detect and image almost any glycoRNA (Soderberg, O., et al., 2006, Nature Methods, 3:995-1000).

The performance of ARPLA was validated in multiple cells with multiple glycoRNA targets then applied to investigate the spatial distribution and intracellular trafficking of glycoRNAs. ARPLA revealed co-localization of glycoRNAs with lipid rafts on plasma membranes and enabled tracking of glycoRNAs through SNARE protein-mediated secretory exocytosis. The abundance of glycoRNA in cancer cell models, was also investigated with ARPLA and, for the first time, the surface glycoRNA differences in cancer progression were revealed. Specifically, in comparison with non-carcinomatous cell (MCF-10A), malignant breast cancer cell (MCF-7) has a much lower glycoRNA abundance and metastatic breast cancer cell (MDA-MB-231) has the lowest glycoRNA signal. These results indicate an inverse correlation between the glycoRNA and tumor malignant degrees. Furthermore, ARPLA was used to monitor the glycoRNA level during innate immune cells differentiation and activation and uncovered its reduction during immune cell differentiation and increase during LPS induced inflammatory responses. The function of glycoRNAs was further examined and it was found that removal of glycoRNA can attenuate the immune cells' attachment to endothelial cells, indicating the potential function of glycoRNA in mediating M-EC interaction during the inflammatory process.

This novel ARPLA method has expanded the toolbox for glycoRNA imaging. Its high sensitivity, selectivity, and ability to image unlabeled glycoRNAs make it a powerful method to discover and investigate other potential roles of glycoRNAs in many biological processes, including cell communication, host-immune response, RNA protein interactions, and, in the process, uncover their relevance to diseases, such as cancer, autoimmune diseases, and inflammations.

Development of ARPLA for In Situ Imaging of glycoRNAs on Cell Surfaces

To visualize glycoRNAs on cell surfaces, ARPLA has been designed to contain four functional components (FIG. 1A): (1) a glycan probe, comprising an aptamer that selectively binds N-acetylneuraminic acid (Neu5Ac), which has been shown to be present in the glycan moiety of glycoRNA, a spacer that avoids steric hindrance during the hybridization, and a DNA linker that serves as a template for subsequent proximity ligation; (2) an RNA binding probe, comprising a DNA that can recognize the RNA moiety of glycoRNA through in situ hybridization (RISH), a spacer, and DNA linkers-Linker R that works together with Linker G; (3) Connectors 1 and 2, that can hybridize with Linkers G and R to allow in situ ligation to generate circular DNA as a template for RCA to produce ssDNA; (4) and a reporter, comprising a short ssDNA that can hybridize with the ssDNA product from the RCA and a fluorophore to report the distribution of glycoRNA on cell surfaces (Yue, H., et al., 2021, Sensors and Actuators B: Chemical, 344:130270; Flynn, R. A., 2021, Cell, 184:3109-3124). Aptamers are single-stranded nucleic acids that act as affinity reagents for different targets, and they have been widely applied for bioimaging, diagnostics, sensors, and therapeutics (Dunn, M. R., et al., 2017, Nature Reviews Chemistry, 1:0076; Sunbul, M., et al., 20201, Nature Biotechnology, 39:686-690; Jou, A. F., et al., 2021, Angewandte Chemie International Edition English, 60:21673-21678; Dey, S. K., et al., 2022, Nature Chemical Biology, 18:180-190; Coonahan, E. S., et al., Science Translational Medicine, 13:eabe1535; Peinetti, A. S., 2021, Science Advances, 7: eabh2848; Wu, L., et al., 2021, Chemical Reviews, 121:12035-12105; Arroyo-Curras, N., et al., 2017, Proceedings of the National Academy of Sciences USA, 114:645-650; Nakatsuka, N., et al., 2018, Science, 362:319-324; Cheung, Y. W>, et al., 2020, Proceedings of the National Academy of Sciences USA, 117:16790-16798; Zhang, Z., et al., 2021, Angewandte Chemie International Edition English, 60:24266-24274; Gunaratne, R., et al., 2018, Nature Biotechnology, 36:606-613; Valero, J., et al., 2021, Proceedings of the National Academy of Sciences, 118: e2112942119; Zhang, Y., et al., 2021, Proceedings of the National Academy of Sciences USA, 118: e2022830118; Zchmitz, A., et al., 2021, Angewandte Chemie International Edition English, 60:10279-10285). As shown in FIG. 1B, the detection of glycoRNAs occurs only when the dual recognition of glycan and RNA (step 1) triggers connectors' hybridization (step 2) and in situ ligation (step 3). Then the intact circular DNA serves as the template for rolling circle amplification (RCA) to generate signal outputs by binding fluorophore-labeled oligonucleotides (step 4).

To validate this method, the binding affinity (K_{d}) of Neu5 Ac aptamer was first determined by isothermal titration calorimetry (ITC) as 91 nM (FIGS. 2A and 2B), which is consistent with the reported K_{d} of 75 nM using target-immobilized magnetic beads method (Yue, H., et al., 2021, Sensors and Actuators B: Chemical, 344:130270). As such, Neu5Ac aptamer has a stronger binding affinity than other glycan-binding reagents, such as lectin (K_{d}=1−10 μM) and antibody (K_{d} in the micromolar range), making ARPLA a sensitive method to detect glycoRNA (Cummings, R. D., et al., 2009, Essentials of Glycobiology 2^{nd} Edition, Chapter 45: Antibodies and Lectins in Glycan Analysis; Tommasone, S., et al., 2019, Chemical Society Reviews, 48:5488-5505). The existence of glycoRNAs in HeLa cells was verified by RNA blot using a protocol described previously (FIG. 2C), which is performed through metabolic labeling with N-azidoacetylmannosamine-tetraacylated (Ac₄ManNAz), biotinylation via click chemistry, and RNA blot (Yue, H., et al., 2021, Sensors and Actuators B: Chemical, 344:130270). ARPLA was then applied to visualize small nuclear RNA U1 (U1 glycoRNA) as a representative target without cell membrane permeabilization using confocal laser scanning microscopy (CLSM). As shown in FIGS. 2D and 3A, bright signals from ARPLA were observed from the cell surface. The membranes' integrity was confirmed intact by transmission-through dye microscopic analysis (FIG. 2E) (Huang, N., et al., 2020, Genome Biology, 21:225). When the same experiments were performed in the absence of the aptamer, the RISH probe, or the connectors, the ARPLA signal was impaired by 13, 270, or 9 folds, which demonstrated the necessity of all ARPLA components (FIGS. 3A and 3B). In addition, replacing the Neu5 Ac aptamer with a DNA having the same number of nucleotides but a scrambled sequence strongly reduced fluorescence signal by 10 folds, indicating the critical role of the aptamer in ARPLA for glycan recognition.

To find out whether the ARPLA method can detect glycoRNA semi-quantitatively, glycoRNA abundance was manipulated by several approaches. The signals generated by ARPLA were reduced by 88% or 90% upon RNase A or T1 digestion compared with cells without treatment (FIGS. 3C and 3D). These results support that cellular surface RNA is required as one of the prerequisites for ARPLA to generate the signals. Moreover, to verify that the glycan moiety of glycoRNA is necessary for the detection, ARPLA was performed on the cells pretreated with glycosylation inhibitors that disrupt the biosynthesis of glycoRNA (Yue, H., et al., 2021, Sensors and Actuators B: Chemical, 344:130270). Specifically, as shown in FIGS. 3C and 3D, after treating the cells with N-linked Glycosylation Inhibitor 1 (NGI 1), Kifunensine, or Swainsonine for 24 h, compared with the untreated group a significantly decreased fluorescence signal was obtained from ARPLA by 86%, 90% or 91%, respectively, demonstrating ARPLA's specificity for glycan moiety of glycoRNA. Moreover, the live cells were treated with endoglycosidases, such as PNGase-F or α2-3,6,8,9 Neuraminidase A, which have been verified to cleave the glycan moiety of glycoRNA (Yue, H., et al., 2021, Sensors and Actuators B: Chemical, 344:130270). This treatment resulted in a decrease of fluorescence signal in PNGase-F or α2-3,6,8,9 Neuraminidase A-treated cells by 89% or 93% compared with the fluorescence signal from untreated cells (FIGS. 3C and 3D). In contrast, compared with the untreated group, a comparative fluorescence signal can be obtained from cells treated with O-glycosidase (FIGS. 3C and 3D), which cannot cleave glycan from glycoRNA (Yue, H., et al., 2021, Sensors and Actuators B: Chemical, 344:130270). These pharmacological and enzymatic treatment experiments validate the specificity of ARPLA to glycan moiety of glycoRNA. Taken together, ARPLA can be applied for in situ imaging of glycoRNA on the cell surface with high sensitivity and selectivity.

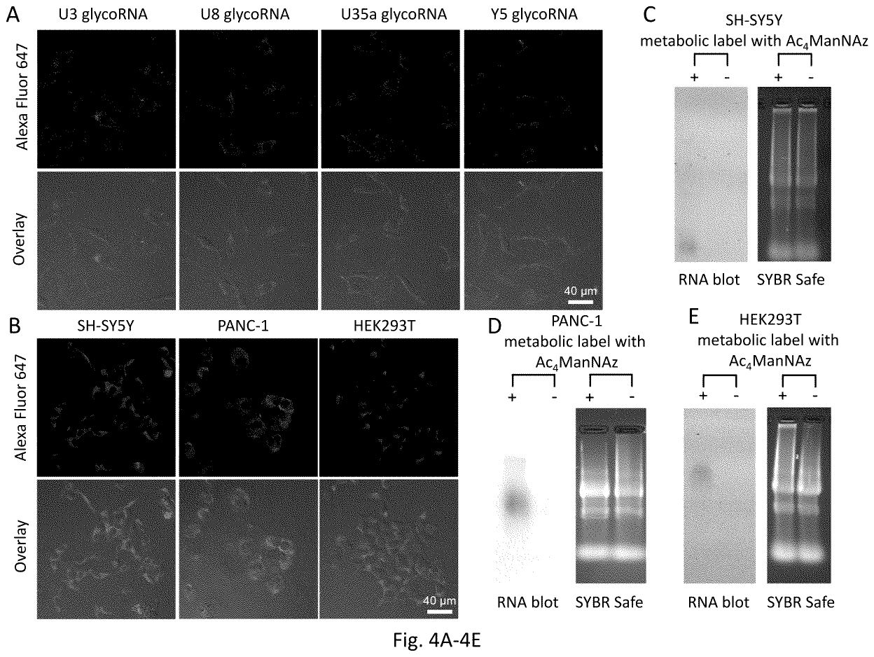

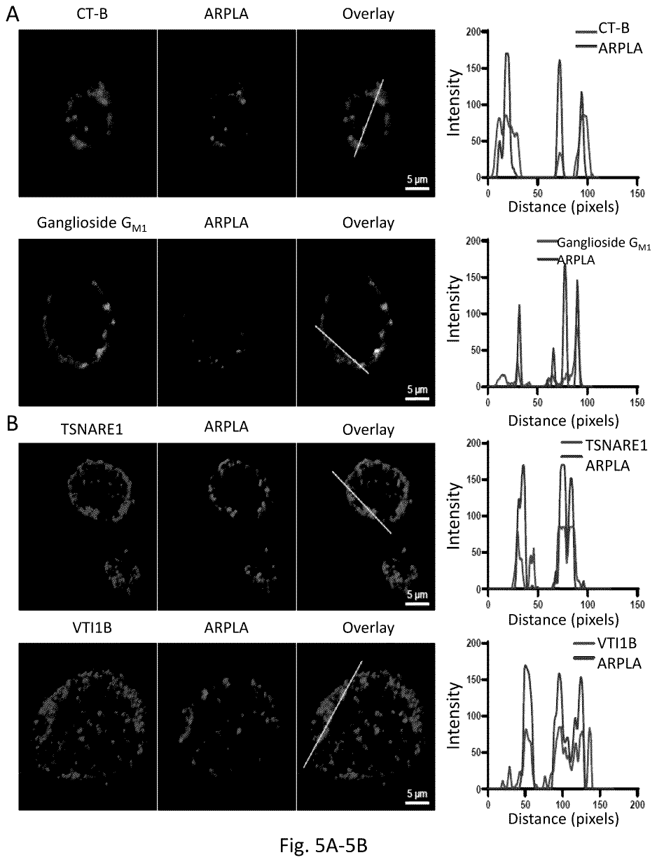

To evaluate the generality of ARPLA, its capabilities to image glycoRNAs were further explored with various RNA sequences and its applications in different cell lines. When the RISH section in the RNA binding probe was replaced with a different sequence to target other glycoRNAs, such as SNORD3a (U3), SNORD8 (U8), SNORD35a (U35a), Y5, ARPLA was able to visualize the glycoRNA with the corresponding sequence in HeLa cells, and not those with a different sequence (FIG. 4A) (Yue, H., et al., 2021, Sensors and Actuators B: Chemical, 344:130270). Furthermore, when ARPLA is applied in different cell lines, including human neuroblastoma cell line (SH-SY5Y), human pancreas carcinoma cell line (PANC-1), and human embryonic kidney cell line (HEK293T), glycoRNA fluorescence signals were detected in all cell lines (FIG. 4B). The presence of glycoRNA was also verified by RNA blot (FIGS. 4C-4E). These results indicate that ARPLA can be applied to visualize glycoRNA with various RNA sequences and in different cell lines.

Application of ARPLA in Visualizing glycoRNA Distribution on Plasma Membranes and Intracellular Trafficking

GlycoRNAs have been reported to present on the plasma membrane, while its detailed spatial distribution on the plasma membrane is still unknown (Flynn, R., et al., 2021, Cell, 184:3109-3124). The plasma membranes are laterally heterogeneous, featuring a variety of distinct subcompartments (Sezgin, E., et al., 2017, Nature Reviews Molecular Cell Biology, 18:361-374). One type of representing examples are lipid rafts, which are ordered and tightly packed microdomains in cell membrane (Sezgin, E., et al., 2017, Nature Reviews Molecular Cell Biology, 18:361-374). Lipid rafts are enriched with saturated phospholipids, sphingolipids, cholesterol, lipidated proteins and glycosylated biomolecules, such as glycolipids, and glycosylphosphatidylinositol (GPI)-anchored proteins (Sezgin, E., et al., 2017, Nature Reviews Molecular Cell Biology, 18:361-374). Inspired by this phenomenon, we are interested to know whether glycoRNAs are also associated with lipid rafts or not. The colocalization of glycoRNAs with lipid rafts was examined by fluorescent imaging. Lipid rafts were visualized using Alexa Fluor 555-labeled cholera toxin subunit B (CT-B) or BODIPY dye-labeled Ganglioside GM, separately. As shown in FIG. 5A, lipid rafts were densely distributed in plasma membranes of the human promyelocytic leukemia cell HL-60, and glycoRNAs signals, indicated by ARPLA, displayed clear colocalization with lipid rafts.

The glycoRNAs represented by ARPLA signals are colocalized with CT-B signal with Pearson's coefficient r=0.67, and the glycoRNAs are also colocalized with Ganglioside G_{M1} signal with r=0.46, indicates the glycoRNAs association with lipid raft.

After visualizing the distribution of glycoRNAs on cell surface, we further utilized the ARPLA to observe the intracellular trafficking of glycoRNAs. During intracellular trafficking and secretory exocytosis, soluble N-ethylmaleimide-sensitive factor attachment protein receptors (SNARE) proteins play an important role in membrane fusion, docking vesicles to target compartments and mediating the fusion of the apposing membranes (Hong, W., 2005, Biochimica et Biophysica Acta Molecular Cell Research, 1744:120-144). Therefore, we studied the association between SNARE proteins and glycoRNAs, which may depict the intracellular trafficking and secretory exocytosis progress of glycoRNAs. Briefly, intracellular glycoRNAs are revealed by ARPLA after cell membrane permeabilization, while SNARE proteins were stained by the related antibodies. As shown in FIG. 5B, glycoRNAs are colocalized with v-SNARE and t-SNARE proteins, which are associated with the vesicle/container, or the target compartment, respectively (Hong, W., 2005, Biochimica et Biophysica Acta—Molecular Cell Research, 1744:120-144). The colocalization is verified by high Pearson's coefficient, r=0.58 for v-SNARE and r=0.70 for t-SNARE. The results indicated that glycoRNAs are presented on the cell surfaces through the SNARE-mediated secretory exocytosis and membrane fusion.

Application of ARPLA in Visualizing glycoRNAs in Cancer Malignant Transformation

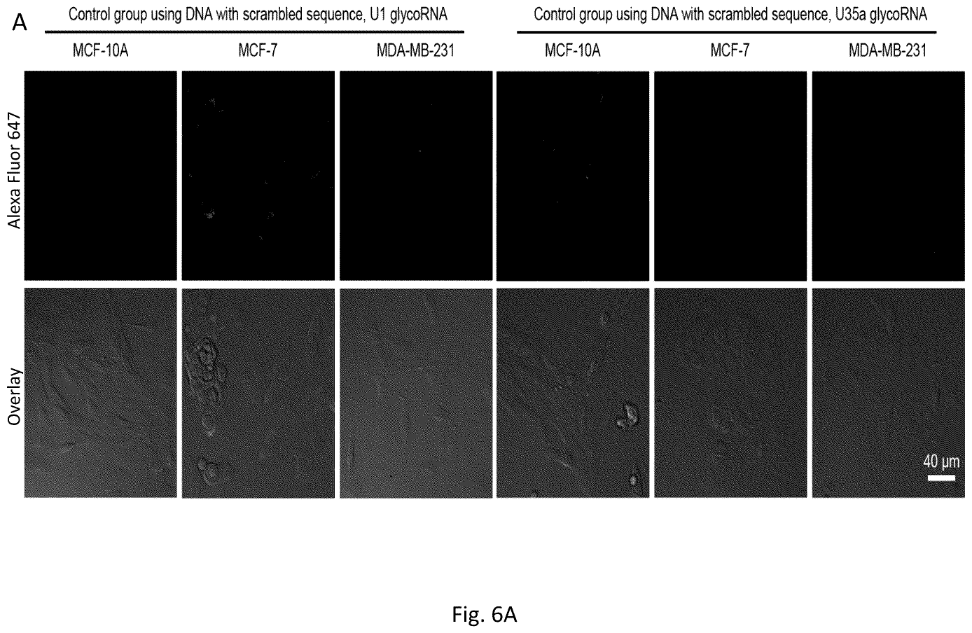

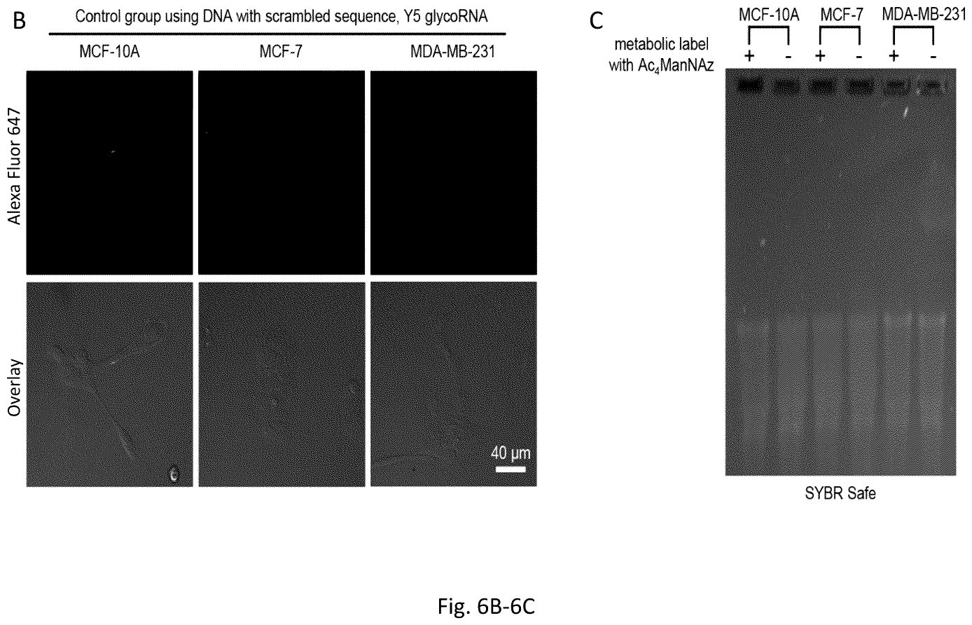

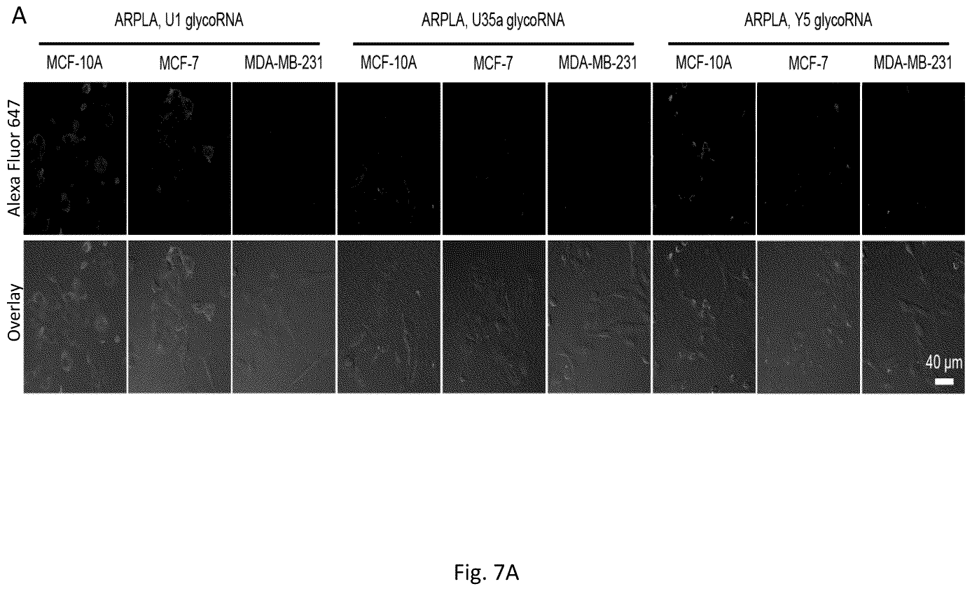

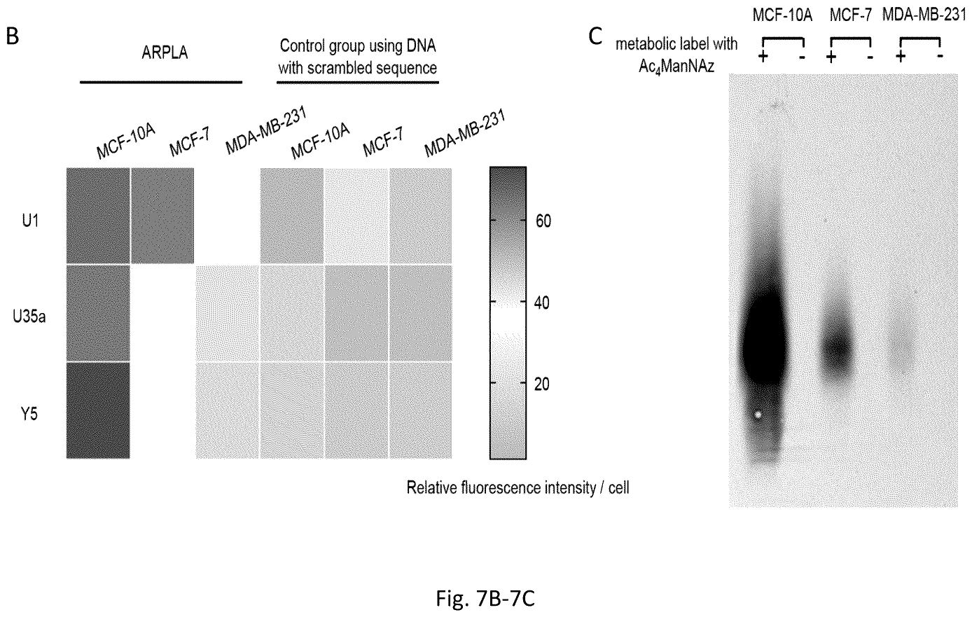

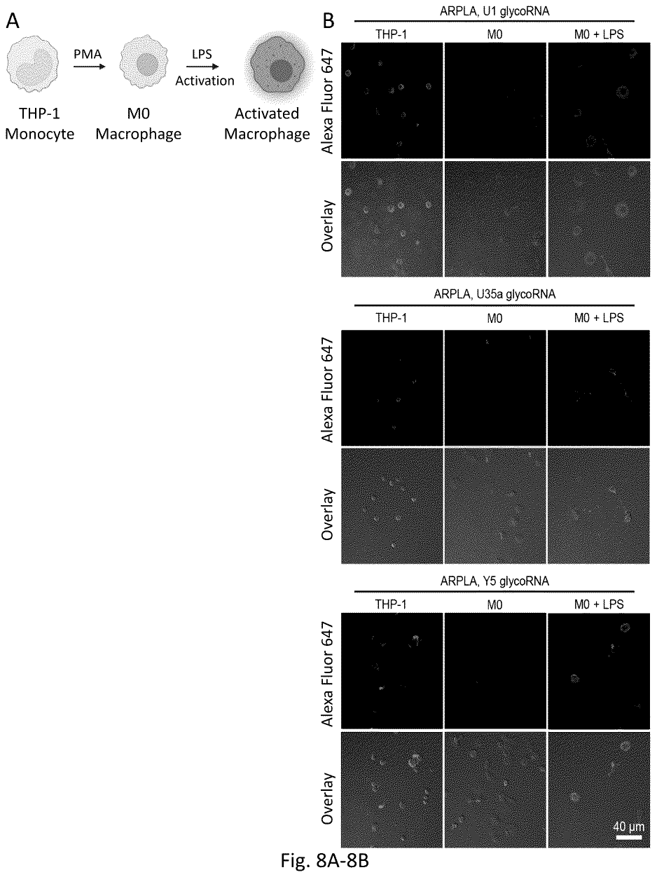

To demonstrate the expansive utility of ARPLA, its application in visualizing glycoRNAs in cancer malignant transformation was examined because an alteration in glycosylation is a hallmark of malignant transformation (Potapenko, I. O., et al., 2015, Molecular Oncology, 9:861-876; Pinho, S. S., et al., 2015, Nature Reviews Cancer, 15:540-555; Munkley, J., et al., 2016, Oncotarget, 7:35478-35489; Pearce, O. M., et al., 2016, Glycobiology, 26:111-128). For instance, progressions of breast cancer from normal cells to non-invasive carcinomas and then metastatic stages are linked with alterations of glycosylation of individual glycoproteins on their cell surfaces and in their extracellular matrix (Potapenko, I. O., et al., 2015, Molecular Oncology, 9:861-876; Reis, C. A., et al., 2010, Journal of Clinical Pathology, 63:322-329; Ma, X., et al., 2016, Cell Death & Disease, 7:e2561). However, the relationship between glycoRNAs and cancer progression is not well understood. To meet this challenge, a series of breast cells were chosen, including non-tumorigenic breast cell line (MCF-10A), malignant breast cell line (MCF-7), and metastatic breast cancer cell line (MDA-MB-231) as representative models for different tumoral status to study the relationship between glycoRNAs and cancer progression. Control experiments were performed utilizing a DNA with scrambled sequences to replace the Neu5Ac aptamer in the Glycan probe to exclude artifacts caused by non-specific binding or hybridization with non-glycosylated RNA, which showed weak fluorescence signals in all breast cell lines (FIGS. 6A and 6B). In contrast, using ARPLA with the correct sequence to target different glycoRNAs, the non-malignant MCF-10A showed the strongest fluorescence signals from U1, U35a, and Y5 glycoRNAs, followed by the malignant MCF-7, while the metastatic MDA-MB-231 showed the weakest fluorescence signals (FIGS. 7A and 7B), indicating that the surface glycoRNA has an inverse correlation to the tumor malignance and tumor metastasis. While a similar trend was observed through RNA blot analysis of the total glycoRNA level (FIGS. 6C and 7C), ARPLA can be performed without metabolic labeling and thus in a shorter time (4 hours vs. RNA blot, which require 24~72 hours metabolic labeling and ~5 hours RNA blot). In addition, ARPLA can reveal the spatial distribution of glycoRNA on the cell membrane and visualize glycoRNA with different sequences, while RNA blot can only study the total glycoRNA level in test tube.

Application of ARPLA to Image glycoRNAs in Innate Immune Cell Differentiation and Responses

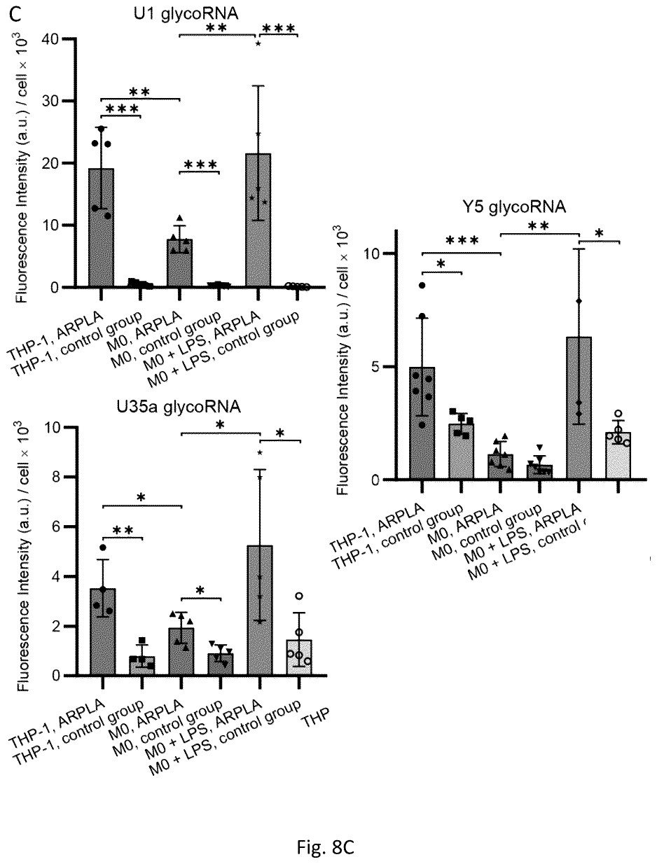

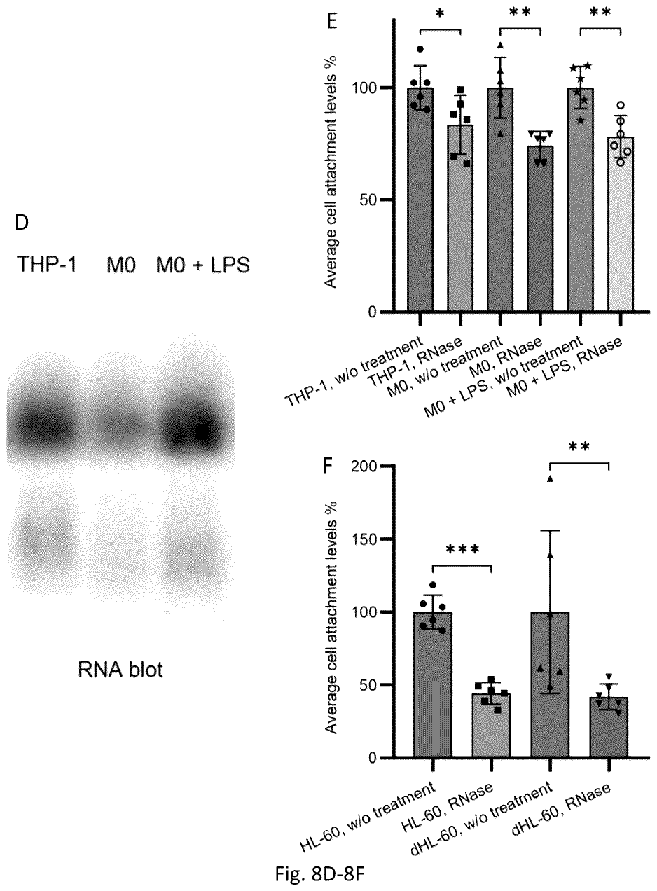

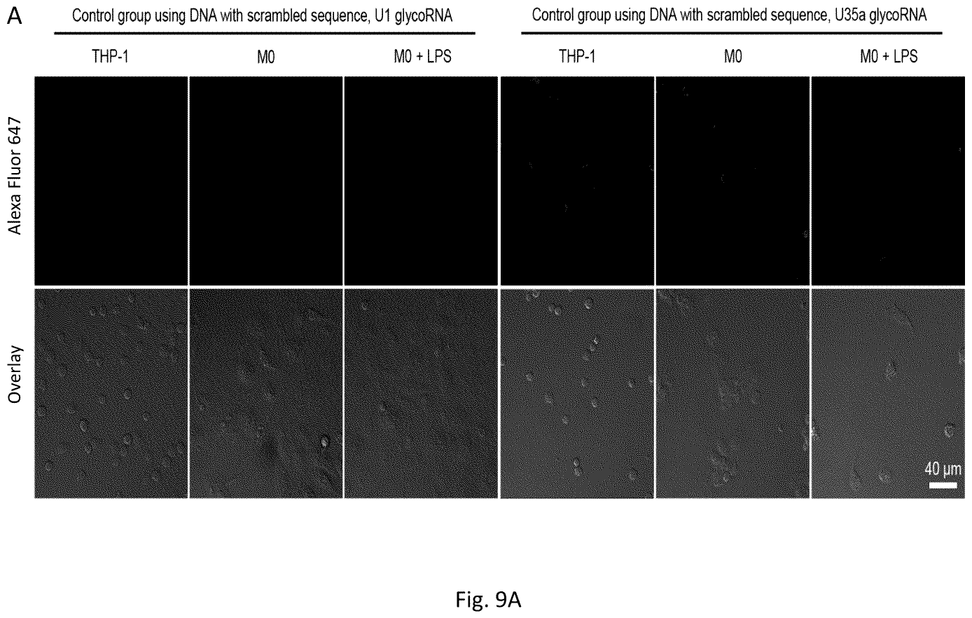

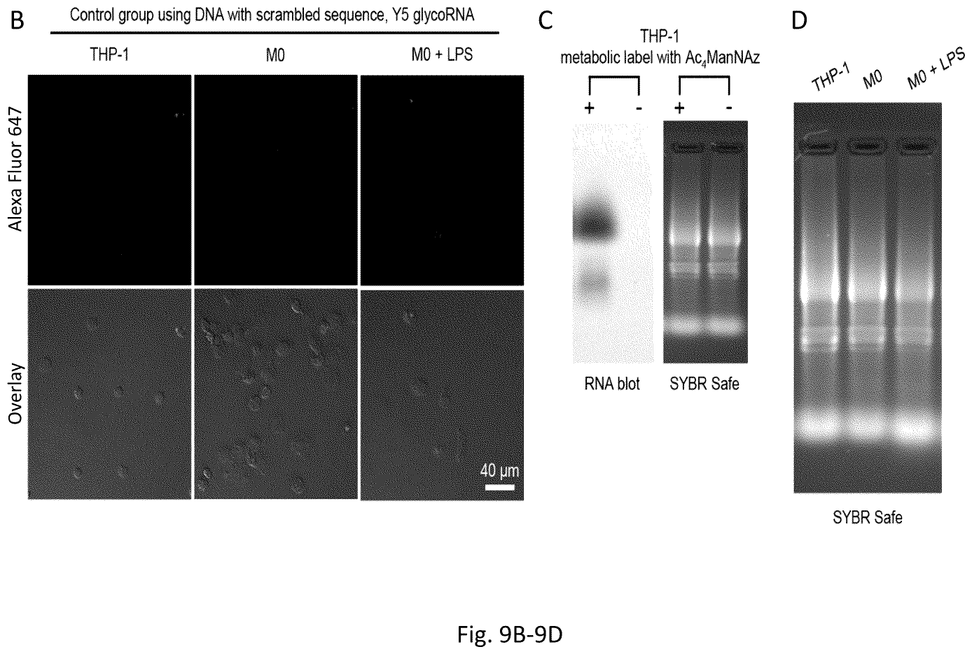

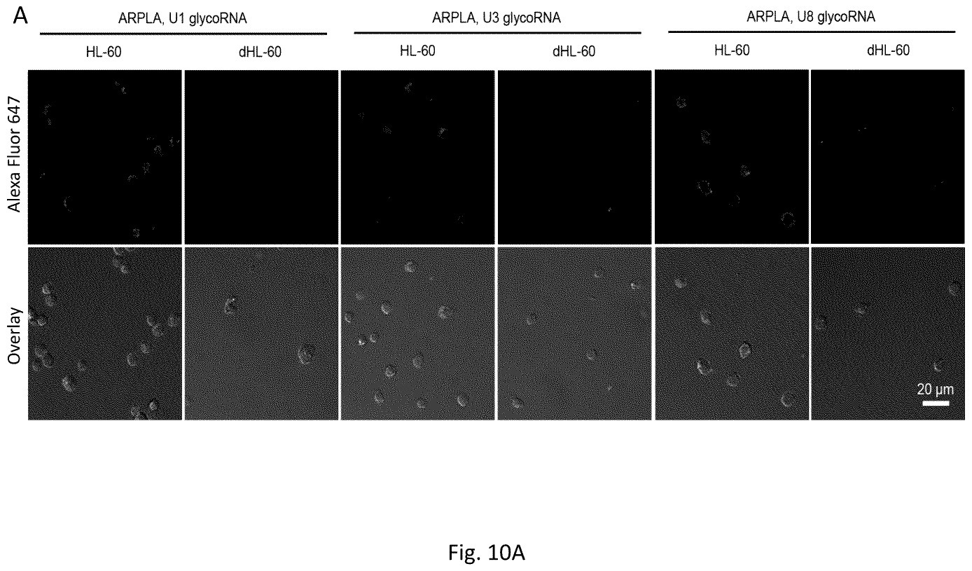

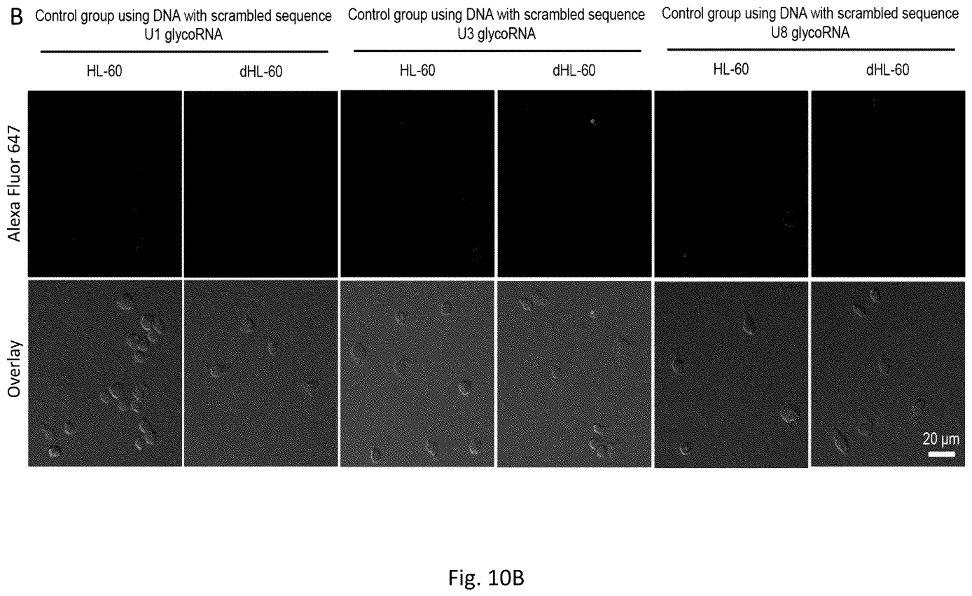

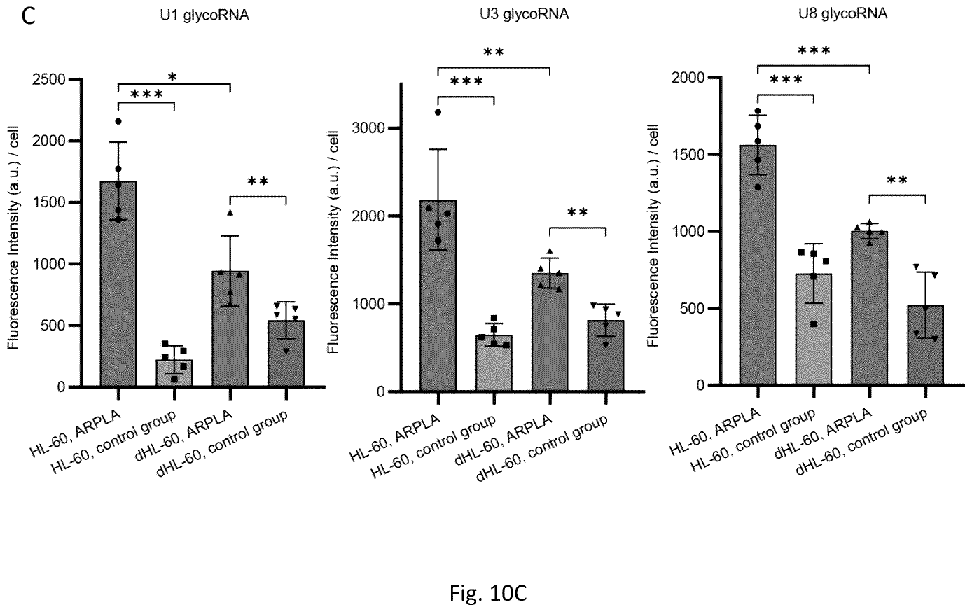

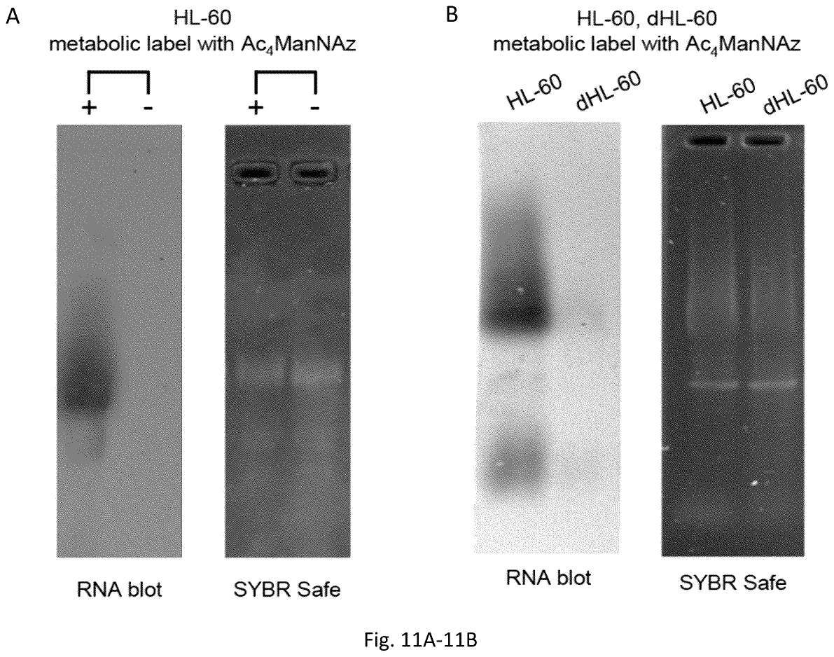

To demonstrate broader applications of ARPLA in other systems, ARPLA was applied to image glycoRNAs in innate immune cell differentiation and responses. It is well known that glycosylation is essential for immune cell development and functions, including those in neutrophils and macrophages, as a timed expression of a series of glycosylated granules (Zhang, T., et al., 2019, Frontiers in Immunology, 10:90; Dusoswa, S. A., et al., 2020, Proceedings of the National Academy of Sciences USA, 117:3693-3703; Bartish, M., et al., 2020, Frontiers in Immunology, 11:564499; Venkatakrishnan, V., et al., 2020, Journal of Biological Chemistry, 295:12648-12660). Previous studies revealed that membrane-associated extracellular RNAs (maxRNA) function by mediating monocyte activities, and glycoRNA could be bound by siglec receptors (Huang, N., et al., 2020, Genome Biology, 21:225; Flynn, R. A., et al., 2021, Cell, 184:3109-3124). Despite these results, the role of glycoRNA in the differentiation and activation of innate immune cells are not well understood. To find out its potential role, THP-1 monocytes were induced to differentiate to the resting macrophage (M0) by phorbol 12-myristate 13-acetate (PMA) and the glycoRNA level in this progress studied via ARPLA. M0 macrophages showed decreased U1, U35a, and Y5 glycoRNA signals than that of THP-1 monocyte (FIGS. 8A-8C, 9A, and 9B). A similar trend was found by the RNA blot experiments (FIGS. 8D, 9C, and 9D). Moreover, a similar trend was also found in the HL-60 neutrophil model. Upon differentiation, mature neutrophils showed a lower glycoRNA level than original HL-60 cells (FIGS. 10 and 11). To gain more insights into the role of glycoRNAs in the innate immune system, changes in glycoRNAs were measured during the immune cells activation process upon pathogenic stimulation. After M0 macrophage activation with E. coli-derived LPS, the activated M0 macrophage showed increase in U1, U35a, and Y5 glycoRNA signals over those of the resting M0 macrophage (FIGS. 8B, 8C, 9A, and 9B). Similar trends in overall glycoRNA level were observed by RNA blot (FIGS. 8D, 9C, and 9D). These results demonstrate that glycoRNAs change significantly during immune cell differentiation and activation, implicating their potential role in innate immune responses.

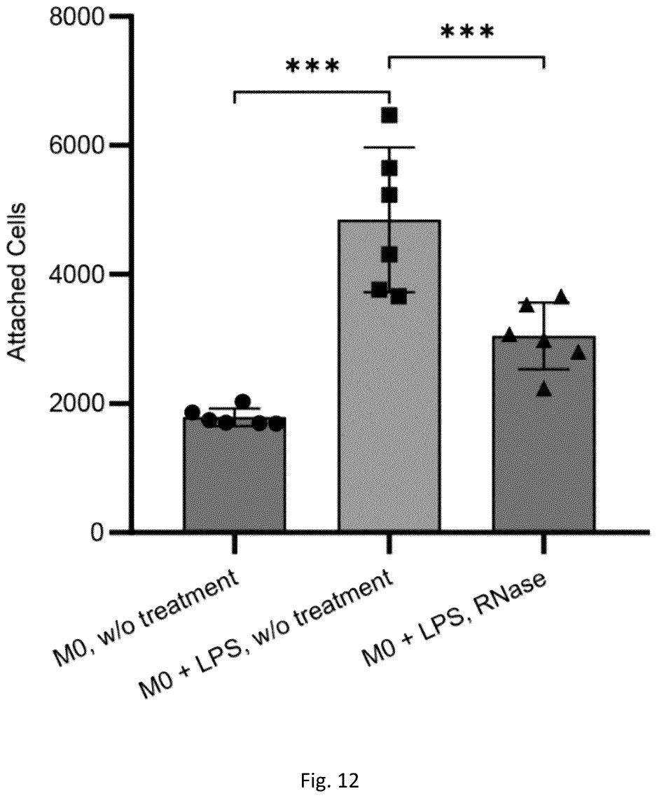

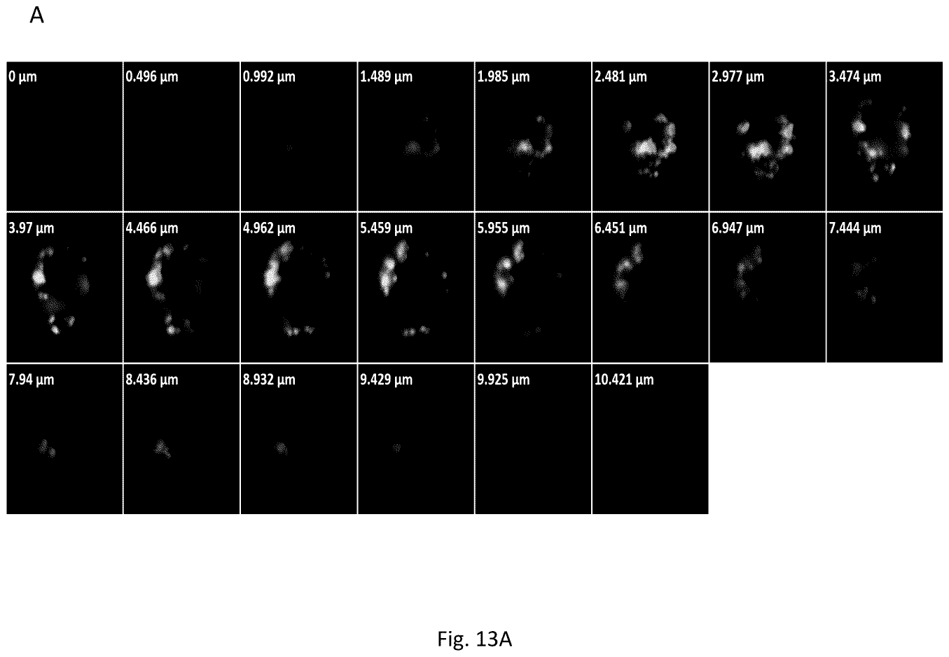

Inspired by the fluctuation of glycoRNA levels during immune cell differentiation and activation, the role that glycoRNAs may play in inflammatory responses was explored. Specifically, it was investigated whether glycoRNAs impact the cellular functions of THP-1 monocytes and macrophages. Monocyte-endothelial cell (M-EC) interaction is one of the essential events in inflammatory responses. For example, the interaction is critical for monocyte migration from the bloodstream into inflammatory tissues in the body and this process was demonstrated to be highly regulated by glycoproteins (Sewald, X., et al., 2015, Science, 350:563-567). A monocyte-EC adhesion assay was adapted to investigate the roles of glycoRNA in M-EC interactions, by comparing the attachment ability of immune cells with that of the RNase-treated cells (Man, S., 2012, Science Translational Medicine, 4:3003197) First, its binding ability to endothelial cells was assessed by incubating THP-1 monocyte, resting M0 macrophage, and activated M0 macrophage with human umbilical vein endothelial cells (HUVEC). As shown in FIG. 8E, THP-1, resting M0, LPS activated M0 macrophage incubated with RNase exhibited a reduced attachment ability by 84%, 74%, 78% compared with no-treatment cells. In consistent with the higher ARPLA signals upon LPS activation, activated M0 exhibited enhanced binding efficiency to HUVEC cells compared with resting M0 macrophage, and such enhancement can be restored by RNase digestion (FIG. 12). Moreover, HL-60, dHL-60 also showed a similar reduction upon RNase digestion (FIG. 8F). These findings suggested that removal of glycoRNA can attenuate these immune cells' attachment to endothelial cells, indicating the function of glycoRNA in mediating M-EC interaction during inflammatory process.

Spatial Distribution of glycoRNAs

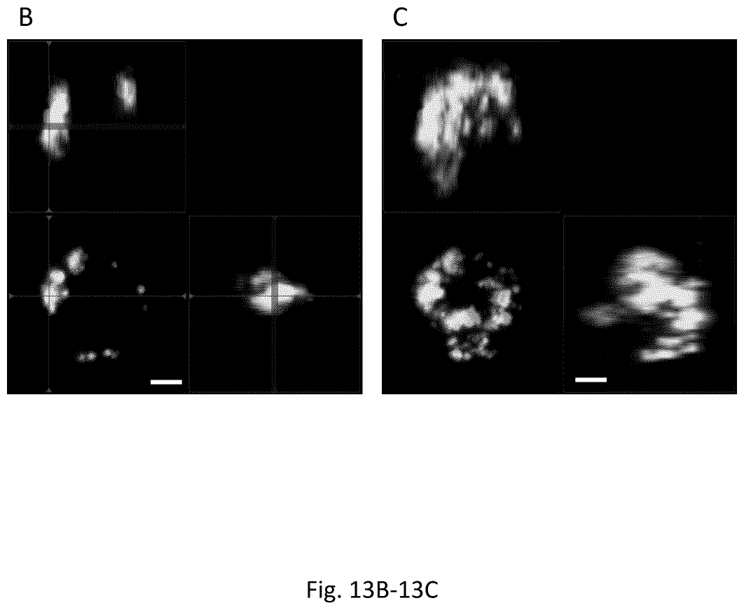

3D Spatial distributions of U1 glycoRNA and CT-B were 3D visualized in HL-60 cells (FIG. 13). Z-stack images were collected with the staining of U1 glycoRNA by ARPLA (green) and lipid raft by CT-B (red). The 3D spatial distributions of U1 glycoRNA were shown in z-slices format (FIG. 13A), orthographic projection (FIG. 13B), and maximum intensity projection (FIG. 13C). As shown in FIG. 13, glycoRNAs signals, indicated by ARPLA, displayed clear colocalization with lipid rafts.

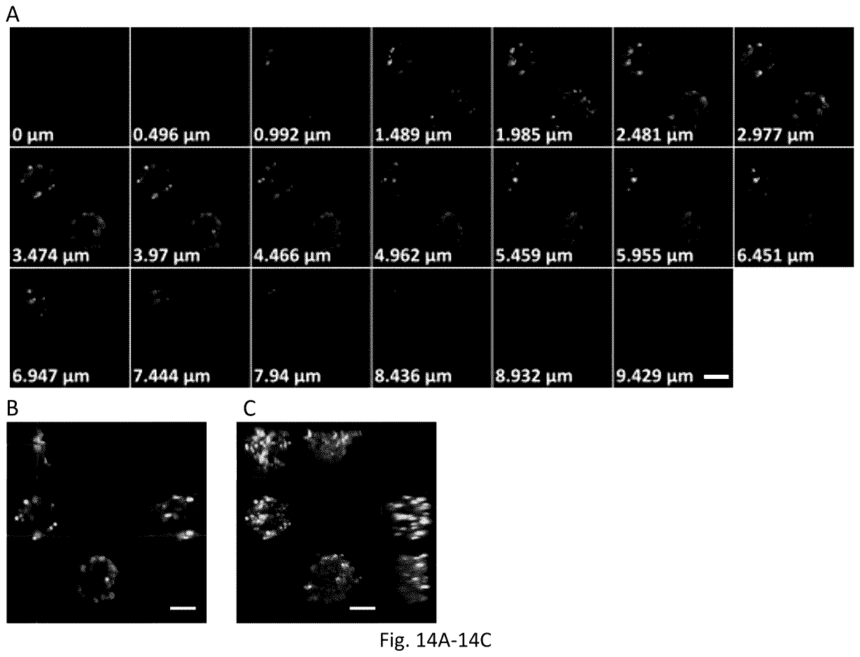

Simultaneous imaging of U1 and Y5 glycoRNAs with multiplex ARPLA in HL-60s, by orthogonal design of different ARPLA sets. U1 (red) and Y5 (green). GlycoRNAs were visualized in Z-stack images presented in z-slices format (FIG. 14A), orthographic projection (FIG. 14B), and maximum intensity projection (FIG. 14C) to show their spatial distributions.

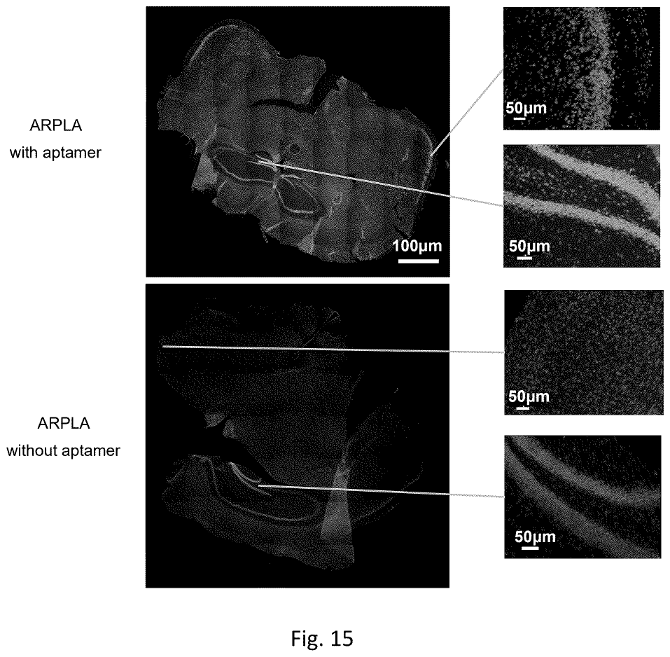

U1 glycoRNA's spatial distribution in the mouse brain was also investigated by ARPLA (FIG. 15). As shown in FIG. 15, glycoRNA has high abundance in hippocampus region in brain.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An assay system for detecting at least one glycosylated molecule, the system comprising:
a) a glycan binding molecule comprising a glycan binding domain, a spacer and a linker domain, wherein the linker domain comprises a first nucleotide sequence specific for binding to a first connector DNA molecule, referred to as “Connector A,” located 5′ and adjacent to a second nucleotide sequence specific for binding to a second connector DNA molecule, referred to as “Connector B;”
b) a target binding molecule comprising a target binding domain specific for binding to a target glycosylated molecule, a spacer and a linker domain, wherein the linker domain comprises a first nucleotide sequence specific for binding to Connector B located 5′ and adjacent to a second nucleotide sequence specific for binding to Connector A;
c) Connector A comprising a phosphorylated 5′ end specific for binding to the linker domain of the glycan binding molecule and a 3′ end specific for binding to the linker domain of the target binding molecule; and
d) Connector B comprising a phosphorylated 5′ end specific for binding to the linker domain of the target binding molecule and a 3′ end specific for binding to the linker domain of the glycan binding molecule.

2. The assay system of claim 1 further comprising a labeled detection molecule.

3. The assay system of claim 1, wherein the target glycosylated molecule is selected from the group consisting of a glycoRNA molecule, a glycoDNA molecule, and a glycopeptide.

4. The assay system of claim 1, wherein the glycan binding domain is selected from the group consisting of a protein, a peptide, a DNA, an RNA, an aptamer, a Siglec, a lectin, antibody, and a chemical ligand.

5. The assay system of claim 1, wherein the glycan-specific binding domain specifically binds to a sialic acid or sugar moiety.

6. The assay system of claim 5, wherein the sialic acid or sugar moiety is selected from the group consisting of N-acetylneuraminic acid (Neu5Ac), N-Glycolylneuraminic acid (Neu5Gc), galactose, glucose and mannose.

7. The assay system of claim 6, wherein the glycan binding domain comprises an aptamer specific for binding to Neu5Ac.

8. The assay system of claim 7, wherein the glycan binding domain comprises the sequence as set forth in SEQ ID NO:1.

9. The assay system of claim 1, wherein at least one of the glycan-specific binding molecule, the target binding molecule, Connector A and Connector B comprises a detectable moiety.

10. The assay system of claim 9, wherein at least one of the glycan-specific binding molecule, the target binding molecule, Connector A and Connector B comprises an emission donor molecule and at least one of the glycan-specific binding molecule, the target binding molecule, Connector A and Connector B comprises an emission acceptor molecule.

11. The assay system of claim 1, comprising a combination of a glycan binding molecule selected from the group consisting of:
a) a glycan binding domain comprising SEQ ID NO:1 and a linker domain as set forth in SEQ ID NO:12, a target binding molecule comprising a linker domain as set forth in SEQ ID NO:15, Connector A comprising a sequence as set forth in SEQ ID NO: 17, and Connector B comprising a sequence as set forth in SEQ ID NO: 18;
b) a glycan binding molecule comprising SEQ ID NO:33, a target binding molecule comprising a linker domain as set forth in SEQ ID NO:21, Connector A comprising a sequence as set forth in SEQ ID NO:22, and Connector B comprising a sequence as set forth in SEQ ID NO:23;
c) a glycan binding molecule comprising SEQ ID NO:33, a target binding molecule comprising a linker domain as set forth in SEQ ID NO:25, Connector A comprising a sequence as set forth in SEQ ID NO:26, and Connector B comprising a sequence as set forth in SEQ ID NO:27; and
d) a glycan binding molecule comprising SEQ ID NO:33, a target binding molecule comprising a linker domain as set forth in SEQ ID NO:29, Connector A comprising a sequence as set forth in SEQ ID NO:30, and Connector B comprising a sequence as set forth in SEQ ID NO:31.

12. The assay system of claim 11 further comprising a labeled detection molecule comprising a sequence selected from the group consisting of: SEQ ID NO:20, SEQ ID NO:24, SEQ ID NO:28, and SEQ ID NO:32.

13-18. (canceled)

19. An assay for detecting at least one glycosylated molecule, the method comprising:
a) contacting a sample with the assay system of claim 1 and
b) detecting the proximity of the Connector A and Connector B molecules.

20-24. (canceled)

25. The assay of claim 19, wherein the method of detecting the proximity of the Connector A and Connector B molecules is selected from the group consisting of ligation followed by rolling circle amplification and probe based detection, ligation followed by PCR based amplification and detection, FRET, Flow cytometry, ligation followed by hybridization chain reaction (HCR), ligation followed by catalytic hairpin assembly (CHA), and ligation followed by detection using DNA-PAINT probes.

26-27. (canceled)

28. The assay of claim 19, the method comprising:
a) contacting the sample with a polymerase for rolling circle amplification of the ligated connector DNA product;
b) contacting the sample with a labeled detection molecule specific for binding to a junction formed from ligation of Connector A and Connector B; and
c) detecting the labeled detection molecule.

29-33. (canceled)

34. The assay of claim 28, wherein the method of detecting the labeled detection molecule is selected from the group consisting of fluorescence detection methods, Flow cytometry, hybridization chain reaction (HCR), catalytic hairpin assembly (CHA), and detection using DNA-PAINT probes.

35-38. (canceled)

39. The assay of claim 28, wherein the DNA ligase comprises T4 DNA ligase.

40. (canceled)

41. The assay system of claim 1, wherein the assay system is a multiplexed assay system, comprising at least two of:
a) a glycan binding molecule comprising a glycan binding domain comprising SEQ ID NO:1 and a linker domain as set forth in SEQ ID NO:12, a target binding molecule comprising a linker domain as set forth in SEQ ID NO:15, Connector A comprising a sequence as set forth in SEQ ID NO:17, Connector B comprising a sequence as set forth in SEQ ID NO:18, and a labeled detection molecule comprising a sequence as set forth in SEQ ID NO:20;
b) a glycan binding molecule comprising a sequence as set forth in SEQ ID NO: 33, a target binding molecule comprising a linker domain as set forth in SEQ ID NO: 21, Connector A comprising a sequence as set forth in SEQ ID NO:22, Connector B comprising a sequence as set forth in SEQ ID NO:23, and a labeled detection molecule comprising a sequence as set forth in SEQ ID NO:24;
c) a glycan binding molecule comprising a sequence as set forth in SEQ ID NO: 33, a target binding molecule comprising a linker domain as set forth in SEQ ID NO: 25, Connector A comprising a sequence as set forth in SEQ ID NO:26, Connector B comprising a sequence as set forth in SEQ ID NO:27, and a labeled detection molecule comprising a sequence as set forth in SEQ ID NO:28; and
d) a glycan binding molecule comprising a sequence as set forth in SEQ ID NO: 33, a target binding molecule comprising a linker domain as set forth in SEQ ID NO: 29, Connector A comprising a sequence as set forth in SEQ ID NO:30, Connector B comprising a sequence as set forth in SEQ ID NO:31, and a labeled detection molecule comprising a sequence as set forth in SEQ ID NO:32.

42. The assay of claim 19, wherein the sample is a biological sample or a patient sample.

43-44. (canceled)

45. A kit comprising a combination of components for performing the assay of claim 40.

46. A method of diagnosing or determining the prognosis of breast cancer in a subject in need thereof, the method comprising
a) obtaining a sample from a patient;
b) contacting the sample with the assay system of claim 1 and
a DNA ligase to ligate Connector A and Connector B;
c) contacting the sample with a polymerase for rolling circle amplification of the ligated connector DNA product;
d) contacting the sample with a labeled detection molecule specific for binding to a junction formed from ligation of Connector A and Connector B;
e) detecting the labeled detection molecule;
f) comparing the level of at least one glycosylated molecule detected in the patient sample to a comparator control level of the glycosylated molecule;
g) diagnosing the subject as having, or at risk of having, breast cancer or having breast cancer with a poorer prognosis when a lower level of at least one glycosylated molecule is detected in the patient sample as compared to the control.

47. The method of claim 46, wherein at least one glycosylated molecule is selected from the group consisting of U1 glycoRNA, U3 glycoRNA, U8 glycoRNA, U35a glycoRNA, and Y5 glycoRNA.

48. (canceled)

49. The method of claim 46, further comprising administering a treatment for the diagnosed breast cancer.

50-53. (canceled)
